(19) United States
(12) Patent Application Publication
(10) Pub. No.: US 2024/0091771 A1
(43) Pub. Date: Mar. 21, 2024

(54) ASSAY DEVICE AND ASSAY METHOD

(71) Applicants: NATIONAL INSTITUTE OF ADVANCED INDUSTRIAL SCIENCE AND TECHNOLOGY, Tokyo (JP); NH FOODS LTD., Osaka-shi, Osaka (JP)

(72) Inventors: Yusuke FUCHIWAKI, Takamatsu-shi, Kagawa (JP); Masato TANAKA, Takamatsu-shi, Kagawa (JP); Shohei YAMAMURA, Takamatsu-shi, Kagawa (JP); Naoki MORISHITA, Tsukuba-shi Ibaraki (JP); Kumiko KAMIYA, Tsukuba-shi Ibaraki (JP); Seiichiro MATSUZAKI, Tsukuba-shi Ibaraki (JP)

(21) Appl. No.: 18/246,143

(22) PCT Filed: Sep. 17, 2021
(86) PCT No.: PCT/JP2021/034332

§ 371 Date: Mar. 21, 2023

(30) Foreign Application Priority Data
Sep. 23, 2020 (JP) 2020-158605

(51) Int. Cl. B01L 3/00

(52) U.S. Cl. CPC B01L 3/502723; B01L 2300/048; B01L 2300/069

(57) ABSTRACT

An assay device and an assay method are each capable of ensuring the accuracy of a target substance detection section. The assay device includes a plurality of assay units 100, each assay unit 100 including a microfluidic channel configured to allow a liquid to flow; a porous absorbing medium disposed at a distance from one end of the microfluidic channel, the one end being located on one side in a flow direction of the liquid; and a separation space disposed between the one end of the microfluidic channel and the porous absorbing medium, in which the microfluidic channel includes, in the microfluidic channel, a detection section 14 having immobilized thereon a substance capable of specifically reacting with a target substance, and an internal standard section 54 having immobilized thereon an internal standard substance, and each assay unit includes two parallel ventilation passages that are respectively adjacent to both sides of the microfluidic channel in the width direction orthogonal to the flow direction, the two parallel ventilation passages communicating with the microfluidic channel to allow for air circulation.

### TECHNICAL FIELD

The present invention relates to an assay device and to an assay method. Specifically, the present invention relates to an assay device and to an assay method, each capable of controlling and ensuring the accuracy of a detection reaction for a target substance.

### BACKGROUND ART

Primarily in the fields of biology, chemistry, and the like, assay devices including microfluidic channels have been employed for performing, for example, inspections, experiments, and assays using very small quantities of liquids, such as reagents, processing agents, and the like on the order of microliters. Among them, an assay device of the lateral flow type is employed for detecting or quantifying the concentration of antibodies or antigens contained in a sample through the ELISA (Enzyme-Linked Immunosorbent Assay) process, immunochromatography, and the like, specifically.

The inventors have previously reported an assay device in which solution exchange occurs in a microfluidic channel when a specimen or a reagent is dropped (for example, see Patent Document 1).

There is known a method for avoiding the influence of measurement errors generated due to Brownian motion of a particulate substance as a measurement target by using an inspection device having formed therein a gap of a recess portion to be filled with a measurement target liquid, and mixing the measurement target liquid with a fluidity reducing material for increasing the viscosity of the liquid (for example, see Patent Document 2).

There is also known a method of, in order to solve the problem of the clogging of a porous body in an immunochromatographic device, providing a long fluidic channel-like space that allows a liquid to automatically flow therethrough by capillary action, thereby effectively improving the visibility and signal strength of a labeling reagent (for example, see Patent Document 3).

There is also known a microchip having an internal liquid reagent, in which a fluid circuit including a space is formed, and it can favorably discharge the reagent from a liquid retention portion when a centrifugal force is applied to the microchip (for example, see Patent Document 4).

### CITATION LIST

### Patent Literature

Patent Document 1: WO 2020/045551 A
Patent Document 2: WO 2016/017591 A
Patent Document 3: JP 2017-78664 A
Patent Document 4: JP 2013-92384 A

### SUMMARY OF INVENTION

### Problem to be Solved by the Invention

In the conventional assay devices, a control line is commonly provided separately from a test line to ensure whether a reaction has occurred each time an assay is performed, so that the accuracy of a signal on the test line is ensured. In addition, to allow a biochemical reaction to occur in a fluidic channel-like space, it is necessary to operate and control the flow of a liquid in the fluidic channel, and to ensure a certain degree of biochemical reaction in the same space, it is necessary to provide an internal standard substance, for example. Specifically, it is necessary to, in the same space, avoid cross-reactivity between reagents, and consider contamination that would occur due to the diffusion of the reagents. Therefore, to realize sequential reactions with the use of an internal standard substance, it is necessary to provide a reactive site for the internal standard substance separately from a reactive site for a measurement target substance, which has not been easy.

Under the control of a laminar flow in a space of a microfluidic channel as in Patent Document 2, only the diffusion of substances contributes to mixing. Thus, the diffusion time is determined in proportion to the square of the diffusion distance. Therefore, the reaction time is short, and measurement errors due to Brownian motion of the measurement target substance are unlikely to occur. In addition, in a reaction space in which a biochemical reaction proceeds, there are many factors that lead to the generation of measurement errors other than the measurement errors due to Brownian motion. However, there is no explicit disclosure of a method for solving such problems.

The technique disclosed in Patent Document 3 is insufficient to confirm whether a reaction has definitely occurred in the fluidic channel and to confirm the presence or absence of a measurement error, as well as the degree of the measurement error. Furthermore, as the microfluidic channel is longer, adsorption to the surface of the fluidic channel is increased, and the internal pressure of the fluidic channel is also increased. Thus, it would be difficult to ensure the same degree of reaction in each fluidic channel for each assay. Thus, the method of Patent Document 3 requires a technique for complicated accuracy control.

The microchip of Patent Document 4 employs a scheme for moving a liquid in the fluid circuit to a desired position using a centrifugal force. Thus, to allow a biochemical reaction to occur, it is necessary to accurately control an air hole for introducing air and internal pressure, which requires complicated operations. In addition, since the microchip has the internal liquid reagent, it is necessary to control the preservation stability of the reagent and the rotating speed of the centrifuge for each reaction. Thus, it would not be easy to control reactions in the fluidic channel. In addition, to control biochemical reactions with various protocols, the method of Patent Document 4 is applicable to general-purpose assays only in a limited range since the types and the number of liquids that can flow are limited.

As described above, among the conventional techniques, there has not been established a highly reliable method for ensuring a certain degree of biochemical reaction in a space of a microfluidic channel. Furthermore, if the length of the microfluidic channel is long, internal pressure of the microfluidic channel is increased, and non-specific adsorption to the surface of the microfluidic channel is also increased. Thus, it would not be easy to adjust the degree of reaction using an internal standard substance for each assay.

### Means for Solving the Problems

The inventors have conducted concentrated studies and arrived at the present invention by developing a technique for disposing in a microfluidic channel an internal standard substance separately from a substance for trapping and reacting with a target substance to be detected, and thus capable of controlling the presence or absence of a reaction of the target substance proceeding in a space of the microfluidic channel, and the accuracy of the reaction.

In other words, an embodiment of the present invention relates to an assay device including a plurality of assay units, each assay unit including a microfluidic channel configured to allow a liquid to flow; a porous absorbing medium disposed at a distance from one end of the microfluidic channel, the one end being located on one side in a flow direction of the liquid; and a separation space disposed between the one end of the microfluidic channel and the porous absorbing medium, in which the microfluidic channel includes, in the microfluidic channel, a detection section having immobilized thereon a substance capable of specifically reacting with a target substance, and an internal standard section having immobilized thereon an internal standard substance, and each assay unit includes two parallel ventilation passages, each adjacent to one of the two sides of the microfluidic channel in a width direction orthogonal to the flow direction, the two parallel ventilation passages communicating with the microfluidic channel to allow for air circulation.

Another embodiment of the present invention relates to an assay method using the foregoing assay device, the method including the following steps that are sequentially performed:
(a) a step of applying a sample to the microfluidic channel;
(b) a step of applying a cleaning solution to the microfluidic channel; and
(c) a step of applying a liquid to the microfluidic channel, the liquid including a first label capable of specifically binding to a target substance, and a second label capable of specifically binding to the internal standard substance.

### Advantageous Effects of Invention

According to the assay device and the assay method of the present invention, it is possible to confirm and control the accuracy of the reaction of the target substance to be detected, using an internal standard substance, and thus perform a highly reliable assay with a simple method. Specifically, in the assay device of the present invention, a specimen and a reagent are flowed through a space of a microfluidic channel based on the stop-and-flow principle so that while a reaction is in progress, the flow of the liquid stops and the liquid thus stays in the space of the fluidic channel. Therefore, even when a reactive site for the internal standard substance is provided near a reactive site for the target substance, the two reactive sites are not substantially affected by the diffusion of the reagent. Furthermore, unlike with the conventional techniques, the length of the microfluidic channel is relatively short, and its structure is simple. Thus, the number and the types of reagents to be flowed through the microfluidic channel are not substantially limited. Therefore, it is possible to ensure the accuracy of the presence or absence of a reaction of the target substance in the space of the microfluidic channel using the assay device just as with the determination of a signal using a control line in immunochromatography.

### BRIEF DESCRIPTION OF DRAWINGS

FIG. 1 is a plan view illustrating an assay device that can be used for a method according to an embodiment of the present invention.

FIG. 2 is an exploded perspective view schematically showing an assay unit corresponding to a single microfluidic channel of the assay device shown in FIG. 1.

FIG. 3 is a plan view schematically showing the assay device shown in FIG. 2.

FIG. 4 is a sectional view taken along line A-A of FIG. 3.

FIG. 5 is a sectional view taken along line B-B of FIG. 3.

FIG. 6 is a sectional view taken along line C-C of FIG. 3.

FIG. 7 is a schematic view schematically showing the states of substances in a microfluidic channel when an assay method according to an embodiment of the present invention is performed.

### MODE FOR CARRYING OUT THE INVENTION

Hereinafter, embodiments of the present invention will be described. However, the present invention is not limited by the following embodiments.

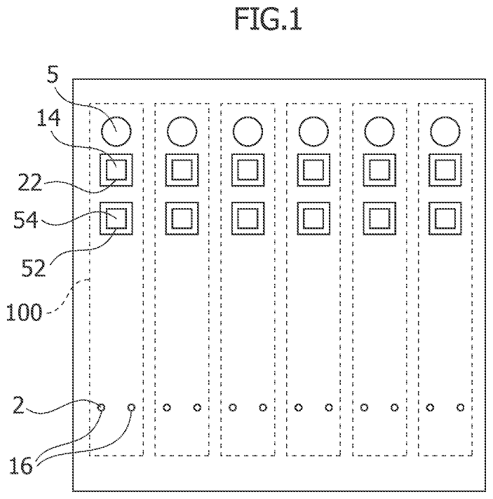

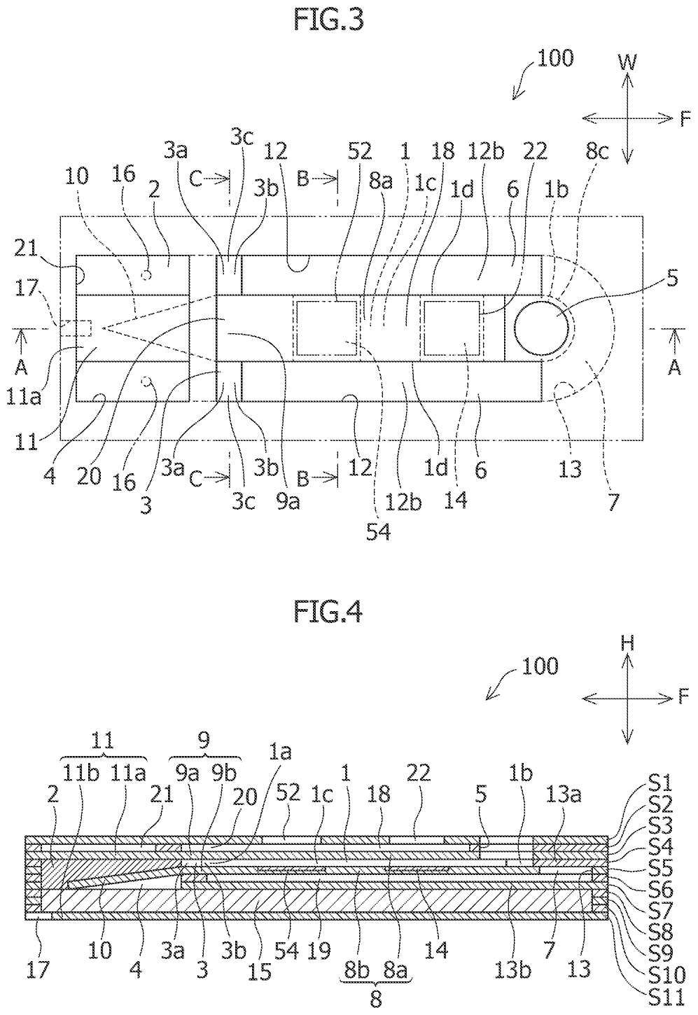

An assay device according to a first embodiment and an assay method according to a second embodiment will be described below. In FIG. 1, the outer shape of the assay device is indicated by a solid line, and the outer shapes of assay units included in the assay device are indicated by phantom lines (that is, broken lines). In FIG. 3, the outer shape of the assay unit is indicated by an imaginary line (that is, a two-dot chain line), and components in the assay unit are indicated by solid lines and phantom lines (that is, broken lines).

The assay device according to the first embodiment allows a liquid to flow through a microfluidic channel in the device, and is used for the assay method according to the second embodiment. The assay method according to the second embodiment is performed for the purpose of detecting, quantifying, or semi-quantifying a target substance that can be contained in a sample. The sample is a hydrophilic liquid that may contain a target substance to be detected. Such a liquid may be a liquid collected from an organism, or a liquid obtained by dissolving a substance collected from an organism in an appropriate solvent. Preferable examples of the liquid include liquid samples derived from an organism, such as whole blood, serum, blood plasma, urine, diluted solutions of feces, saliva, and cerebrospinal fluid of humans or animals. The use of such a sample allows the assay device to effectively diagnostically take measurements of a specimen in the liquid sample for the purpose of testing for pregnancy, urine, feces, adult diseases, allergies, infectious diseases, drugs, or cancers, for example. Other preferable examples of the liquid include suspensions of food, extracts of food, cleaning water used in production lines of food manufacturing factories, wiping solutions, drinking water, river water, and soil-derived suspended solids. The use of such a liquid allows the assay device to measure allergens or pathogens contained in food or drinking water, or contaminants in river water or soil. For the assay method performed with the assay device according to the first embodiment, it is also possible to use various liquids other than samples, for example, reagents for use in assays, such as common biochemical reagents, immunochemical reagents, antibody-related reagents, peptide solutions, protein or enzyme related reagents, cell-related reagents, lipid-related reagents, natural substance or organic compound-related reagents, and carbohydrate-related reagents. However, the present invention is not limited thereto.

In the specification, the “target substance” refers to a substance to be detected or measured. For example, the “target substance” may be saccharides (for example, glucose), proteins or peptides (for example, serum proteins, hormones, enzymes, immunoregulatory factors, lymphokines, monokines, cytokines, glycoproteins, vaccine antigens, antibodies, growth factors, or multiplication factors), fats, amino acids, nucleic acids, steroids, vitamins, pathogens or antigens thereto, natural substances or synthetic chemicals, contaminants, medicines for therapeutic purposes or illegal drugs, metabolites of these substances, their fragments, and those containing antibodies.

In the specification, the “lateral flow” refers to the flow of a liquid moved by gravitational sedimentation as a drive force. The movement of a liquid based on the lateral flow refers to the liquid movement predominantly (mostly) caused by a liquid drive force generated by gravitational sedimentation. In contrast, the movement of a liquid based on a capillary force (a capillary phenomenon) refers to liquid movement predominantly (mostly) caused by interfacial tension. Liquid movement based on lateral flow differs from liquid movement based on capillary force.

In the specification, the “microfluidic channel” refers to a fluidic channel configured to allow a liquid to flow therethrough in the assay device in order to detect or measure a specimen using a very small quantity of liquid on the order of a μL (microliter), that is, in the range of greater than or equal to about 1 μL to less than about 1 ml (milliliter), or in order to weigh a very small quantity of liquid.

In the specification, “film” refers to a membranous or plate-like object with a thickness of less than or equal to about 200 μm (micrometers), and the “sheet” refers to a membranous or plate-like object with a thickness of greater than about 200 μm.

In the specification, “plastic” refers to a polymerized or shaped material produced using a polymerizable or polymer material as an essential component. The plastic may include polymer alloys formed by combining two or more types of polymers.

In the specification, the “porous medium” refers to a member having a number of micropores and capable of attracting liquid and allowing liquid to flow through it, for example, paper, cellulose membranes, non-woven fabric, or plastic. The “porous medium” may exhibit a hydrophilic property when a hydrophilic liquid is used, and may exhibit a hydrophobic property when a hydrophobic liquid is used. Specifically, the “porous medium” may exhibit a hydrophilic property, and may be formed as paper. Furthermore, the “porous medium” may be formed as any one of cellulose, cellulose nitrate, cellulose acetate, filter paper, tissue paper, toilet paper, paper towel, fabric, or a hydrophilic porous polymer that is permeable to water.

### First Embodiment: Assay Device

An embodiment of the present invention relates to an assay device. The assay device according to the present embodiment includes a plurality of assay units, and each assay unit includes a microfluidic channel configured to allow a liquid to flow; a porous absorbing medium disposed at a distance from one end of the microfluidic channel, the one end being located on one side in a flow direction of the liquid; and a separation space disposed between the one end of the microfluidic channel and the porous absorbing medium, in which the microfluidic channel includes, in the microfluidic channel, a detection section having immobilized thereon a substance capable of specifically reacting with a target substance, and an internal standard section having immobilized thereon an internal standard substance, and each assay unit includes two parallel ventilation passages, each of which is adjacent to one of the two sides of the microfluidic channel in the width direction orthogonal to the flow direction, the two parallel ventilation passages communicating with the microfluidic channel to allow for air circulation.

### Outline of Schematic Structure of Assay Device

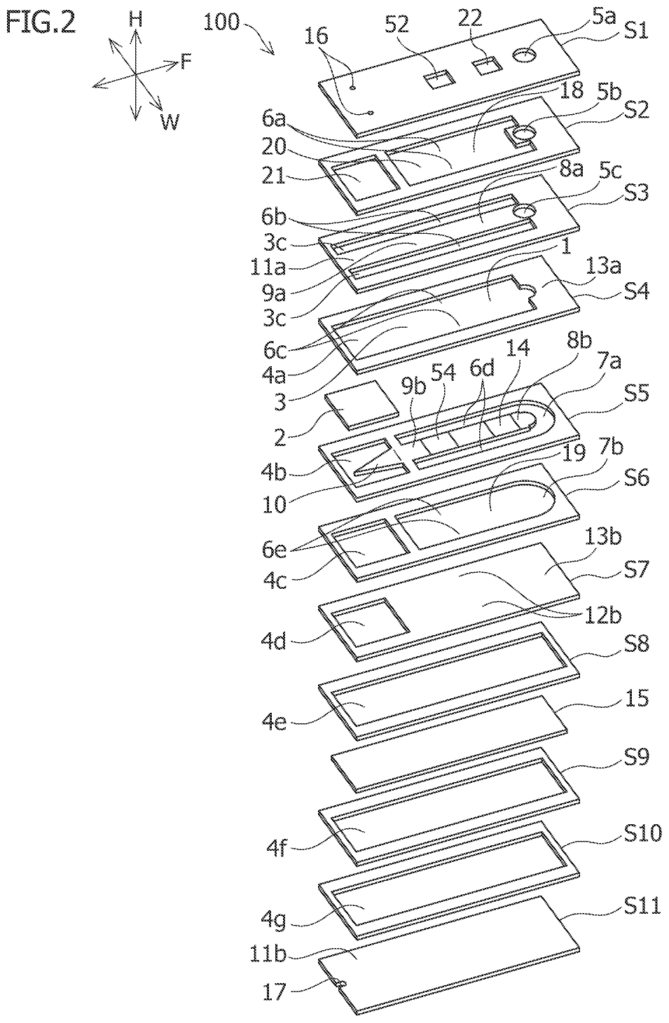

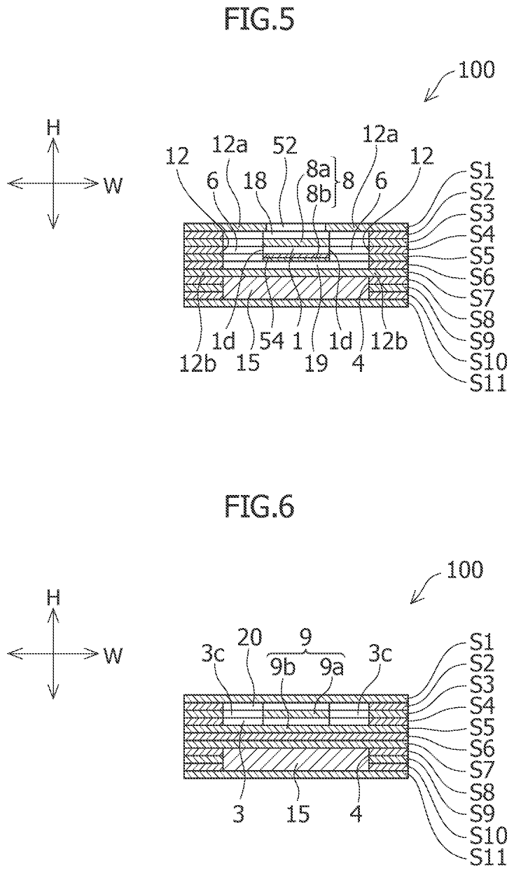

First, the schematic structure of the assay device according to the present embodiment will be described with reference to FIGS. 1 to 6. As shown in FIG. 1, the assay device includes two or more assay units 100. Each assay unit 100 includes an inlet 5 through which a liquid containing a sample and a cleaning solution can be supplied, and a microfluidic channel (not shown in FIG. 1) configured to allow the liquid to flow therethrough. The microfluidic channel includes a detection section 14 and an internal standard section 54 that are visible from outside. Hereinafter, the schematic structure of each assay unit will be described with reference to FIGS. 2 to 6. As described below, a direction along a liquid flow (indicated by an arrow F) in the microfluidic channel 1 will be referred to as “flow direction”. In the present embodiment, a liquid flows toward one side from the other side of the microfluidic channel 1. Therefore, the one side in the flow direction is defined as the downstream side, and the other side in the flow direction is defined as the upstream side.

The assay unit 100 includes a first porous absorbing medium 2 disposed at a distance from one end 1a of the microfluidic channel 1 located on the one side (that is, the downstream side) in the flow direction. The assay unit 100 also includes a separation space 3 disposed between the one end 1a of the microfluidic channel 1 and the first porous absorbing medium 2. The separation space 3 is in the form of a cavity in the assay unit 100. The first porous absorbing medium 2 is configured to be able to absorb a liquid from the one end 1a of the microfluidic channel 1. The assay unit 100 also includes a housing space 4 capable of housing the first porous absorbing medium 2. The housing space 4 is formed to be continuous with the separation space 3 in the flow direction.

The assay unit 100 also includes the inlet 5 disposed at other end 1b of the microfluidic channel 1 located on the other side (that is, the upstream side) in the flow direction. The inlet 5 is configured to allow a liquid to be supplied to the microfluidic channel 1. In the microfluidic channel 1, a liquid supplied from the inlet 5 flows from the other end 1b to the one end 1a via an intermediate section 1c between the one end 1a and the other end 1b.

The assay unit 100 includes two parallel ventilation passages 6, each of which is adjacent to one of the two sides of the microfluidic channel 1 in the width direction (indicated by arrow W) substantially orthogonal to the flow direction. Each parallel ventilation passage 6 is configured to allow for air circulation. The microfluidic channel 1 communicates with the two parallel ventilation passages 6 in the width direction. Each of the parallel ventilation passages 6 extends along the flow direction. Specifically, each of the two parallel ventilation passages 6 may extend along one of two side edges 1d of the microfluidic channel 1 in the width direction.

The assay unit 100 further includes a connecting ventilation passage 7 that connects the two parallel ventilation passages 6 and extends around the inlet 5. The connecting ventilation passage 7 is also configured to allow for air circulation. The air is circulated through the two parallel ventilation passages 6 and the connecting ventilation passage 7 that run through each other. Other ends of the two parallel ventilation passages 6 located on the other side in the flow direction may be connected to the connecting ventilation passage 7. The assay unit 100 may be configured to include no connecting ventilation passage.

The assay unit 100 includes a microfluidic channel wall 8 that defines the microfluidic channel 1. The microfluidic channel wall 8 includes a top portion 8a and a bottom portion 8b that are respectively located on the top side and the bottom side in the height direction (indicated by an arrow H) substantially orthogonal to the flow direction and the width direction. The top portion 8a and the bottom portion 8b of the microfluidic channel wall 8 are held at a distance from each other in the height direction. The distance between the top portion 8a and the bottom portion 8b in the height direction is determined to allow, when a liquid flows through the microfluidic channel 1, interfacial tension of the liquid to be generated for preventing the leakage of the liquid to the parallel ventilation passages 6. The microfluidic channel 1 opens to both sides of its width into the two parallel ventilation passages 6.

The assay unit 100 includes a separation space wall 9 that defines the separation space 3 in cooperation with the first porous absorbing medium 2. The separation space may be further defined by other components in addition to the first porous absorbing medium and the separation space wall. The separation space wall 9 includes a top portion 9a and a bottom portion 9b that are respectively located on the top side and the bottom side in the height direction.

The assay unit 100 includes a guide wall 10 protruding toward one side in the flow direction in the housing space 4 from the bottom portion 9b of the separation space wall 9. The guide wall 10 abuts the first porous absorbing medium 2 in the height direction. The bottom portion 9b of the separation space wall 9 and the guide wall 10 are inclined to be away from the microfluidic channel 1 in the height direction from the other side to one side in the flow direction. Note that FIG. 4 does not clearly show the inclination of the bottom portion 9b of the separation space wall 9 as it tends to be smaller than that of the guide wall 10. However, the guide wall may be formed to protrude toward the one side in the flow direction in the housing space from the top portion of the separation space wall. In such a case, the top portion of the separation space wall and the guide wall may be formed to be away from the microfluidic channel in the height direction from the other side to the one side in the flow direction.

The assay unit 100 includes a housing space wall 11 that defines the housing space 4. The assay unit 100 includes two parallel ventilation passage walls 12 that respectively define the two parallel ventilation passages 6. The assay unit 100 also includes a connecting ventilation passage wall 13 that defines the connecting ventilation passage 7.

The assay unit 100 includes the detection section 14 disposed in the intermediate section 1c of the microfluidic channel 1. The detection section 14 is a section in which a substance capable of specifically reacting with a target substance that may be present in a sample is immobilized. The target substance can be appropriately determined according to the purpose of the assay method, and it is not limited to a specific substance. The substance configured to be able to specifically react with the target substance is determined in consideration of the relationship with the target substance. Such a substance may be, when the target substance is an antigen, an antibody that specifically binds to the antigen, and may be, when the target substance is an antibody, an antigen that specifically binds to the antibody. In the following description of the specification, the substance configured to be able to specifically react with the target substance may be collectively referred to as a detecting antibody. However, the substance in the present invention is not limited to an antibody.

The detection antibody may be directly immobilized in the microfluidic channel 1 or may be immobilized on a porous reaction medium disposed in the microfluidic channel 1, or it may be bound to both. The reaction porous medium may be, for example, cellulose that supports an antibody or an antigen, but it is not limited to a specific type of porous medium. In any case, it is preferable that a signal derived from the target substance that has bound to the detection antibody be immobilized in a form observable from the outside of the assay unit 100. In an aspect, the detection antibody is preferably immobilized at the position of the bottom portion 8b of the microfluidic channel 1. In another aspect, the detection antibody is preferably immobilized at the position of the top portion 8a of the microfluidic channel 1. The detection antibody located at the top portion 8a can be observed from the outside if the top portion is formed of a transparent material. A region in which the detection section 14 is provided may have any shape and area that allows a signal derived from the target substance to be viewed or detected, and such a shape and area are not limited to specific ones. For example, the detection section 14 may be provided by immobilizing the detection antibody on a quadrangular region having sides with the same length as the width of the microfluidic channel 1. When a window portion 22, described below, is provided, the detection section 14 may be provided by immobilizing the detection antibody on a region in the bottom portion 8b or in the top portion 8a of the microfluidic channel 1, corresponding to the shape and the area of at least the window portion.

Although the assay unit 100 shown in the drawings includes one detection section 14, the assay unit 100 may include two or more detection sections. For example, when the assay unit 100 includes a first detection section and a second detection section, the first detection section may have a first detection antibody, and the second detection section may have a second detection antibody different from the first detection antibody. This allows for the simultaneous detection of a first target substance that specifically binds to the first detection antibody, and a second target substance that specifically binds to the second detection antibody.

The assay unit 100 also includes the internal standard section 54 disposed in the intermediate section 1c of the microfluidic channel 1. The internal standard section 54 has an internal standard substance immobilized thereon. The internal standard substance is not affected by the presence of the target substance or the amount of the target substance, and it functions as a substance that provides an index of a predetermined reaction progress in the microfluidic channel. Thus, the internal standard substance may be a substance that does not react with the target substance but can specifically react with a label. Alternatively, the internal standard substance may be a substance that does not react with the target substance but can specifically react with a substance that does not interfere with a reaction of the target substance. The substance that does not interfere with a reaction of the target substance is also referred to as a non-inhibitor. Herein, the label refers to a substance that can specifically bind to the target substance and the internal standard substance, and generate a signal based on the target substance and the internal standard substance. Examples of the signal that can generate a signal include not only a substance that generates a signal alone, but also a substance that generates a signal with the addition of other substances, such as a substrate. Thus, the label is a molecule having both a portion that commits to specific binding, and a portion that commits to generating a signal. Examples of the label include a dye, a fluorescent material, and an enzyme-bound antibody or antigen. In addition, the non-inhibitor may be a pH indicator or a dye, for example. The internal standard substance can be determined in consideration of the relationship with the target substance, the label, and the non-inhibitor, and may be an antibody, an antigen, a secondary antibody produced from an animal antibody, a pH indicator, or a dye, for example. The label and the non-inhibitor do not form the assay unit, and are used by being applied to the microfluidic channel 1 in the assay method described below. The type of the label may be one, but it is also possible to use a first label and a second label that are of different types. Details will be described below.

The internal standard substance may also be directly immobilized on the microfluidic channel 1 or may be immobilized on a porous reaction medium disposed on the microfluidic channel 1, or it may be bound to both. In any case, it is preferable that a signal derived from the internal standard substance be immobilized in a form observable from the outside of the window portion 52. Thus, the internal standard substance is preferably immobilized at the position of the bottom portion 8b or the top portion 8a of the microfluidic channel 1. The internal standard substance located at the top portion 8a can be observed from the outside if the top portion 8a is formed of a transparent material. The shape and the area of a region in which the internal standard section 54 is provided may be similar to those of the detection section 14. The shape and the area of the region in which the internal standard section 54 is provided may be either the same as, or be different from, those of the detection section 14.

In FIGS. 1 to 4, the internal standard section 54 is provided on the downstream side of the microfluidic channel 1 at a distance from the detection section 14 in the flow direction. The distance between the internal standard section 54 and the detection section 14 is preferably about equal to or greater than the width of the microfluidic channel 1. This is because if the internal standard section 54 and the detection section 14 are located close to each other, it may be impossible to clearly distinguish a signal from the internal standard section 54 from a signal from the detection section 14 when the assay method is performed. Although it is not shown, the positional relationship between the internal standard section and the detection section may be reversed. That is, the internal standard section may be provided on the upstream side of the microfluidic channel 1 close to the inlet 5. In such a case also, the distance between the internal standard section 54 and the detection section 14 is preferably about equal to or greater than the width of the microfluidic channel 1. To measure a sample that can contain a high concentration of a target substance, it may be preferable to provide the internal standard section on the upstream side of the microfluidic channel 1 close to the inlet 5. This is because a signal from the internal standard section on the upstream side is unlikely to be affected by a signal from the detection section on the downstream side. The high concentration differs depending on the type of the target substance, and it is not limited to a specific concentration. In an assay device including two or more detection sections, the internal standard section may be provided on the downstream side or the upstream side of the two or more detection sections, or the internal standard section may be provided at an intermediate position between the two or more detection sections.

A combination of the substance configured to be able to specifically react with the target substance (that is, the detection antibody), the internal standard substance, and the label may differ depending on the target substance and the aspect of detection. In addition, regarding the label, the first label that can specifically react with the target substance, and the second label that can react with the internal standard substance may be either different or be the same. When the target substance is an allergen, the substance configured to be able to specifically react with the target substance may be an anti-allergen antibody, and the internal standard substance may be an antibody produced by a species of animal. Examples of the species of animal include, but are not particularly limited to, mammals such as humans, pigs, goats, mice, rats, and rabbits, and birds such as chickens. For example, when the internal standard substance is an anti-mouse IgG antibody, the label may be a horseradish peroxidase (HRP)-labeled anti-allergen antibody (derived from a mouse). As another example, when the target substance is an allergen, the substance configured to be able to specifically react with the target substance may be an anti-allergen antibody, and the internal standard substance may be an antibody produced by a species of animal. In such a case, it is possible to use the first label and the second label as the labels, and the first label may be an HRP-labeled anti-allergen antibody (that is, an antibody produced from different animal species from the internal standard substance), and the second label may be an HRP-labeled animal antibody (that is, an antibody produced from the same animal species as the internal standard substance). For example, when the internal standard substance is an anti-rabbit IgG antibody, the first label may be an HRP-labeled anti-allergen antibody produced from animal species other than rabbits, and the second label may be an HRP-labeled rabbit antibody. Other examples of the combination of the target substance, the substance configured to be able to specifically react with the target substance, the internal standard substance, and the label include a combination of a nucleic acid probe and an aptamer. In addition, a pH indicator in particular can be immobilized as an internal standard substance and a mechanism can be used which, when a predetermined solution such as a diluent or cleaning solution passes therethrough, undergoes a color change due to the pH of the solution. However, the combination of such substances used in the present invention is not limited thereto.

### Detailed Structure of Assay Unit

The detailed structure of the assay device according to the present embodiment will be described with reference to FIGS. 2 to 6. Such an assay device may be further configured as follows. The assay unit 100 may have its height direction vertically directed in the state of use. In such a case, the top portion and the bottom portion of the assay unit 100 respectively face upward and downward in the vertical direction.

The microfluidic channel 1 is formed substantially linearly. However, in the present invention, the microfluidic channel may also be formed in a curved or bent shape. The other end 1b of the microfluidic channel 1 is defined by the other end 8c of the microfluidic channel wall 8. The other end 8c of the microfluidic channel wall 8 is located between the microfluidic channel 1 and the connecting ventilation passage 7.

For example, the height of the microfluidic channel 1, that is, the distance in the height direction between the top portion 8a and the bottom portion 8b of the microfluidic channel wall 8 may be in the range of about 1 μm to about 1000 μm (that is, about 1 mm (millimeter)), inclusive. The width “d” of the microfluidic channel 1 may be in the range of about 100 μm to about 10000 μm (that is, about 1 cm (centimeter)), inclusive, for example. The length of the microfluidic channel 1 in the flow direction may be in the range of about 10 μm to about 10 cm, inclusive, for example. The volume “P” of the microfluidic channel 1 may be in the range of about 0.1 μL to about 1000 μL, inclusive, or more preferably, in the range of greater than or equal to about 1 μL to less than about 500 μL, for example. However, the respective dimensions and the volume of the microfluidic channel are not limited to these.

The height of the first porous absorbing medium 2 is greater than that of the microfluidic channel 1. The first porous absorbing medium 2 protrudes in the height direction closer to the bottom side than the microfluidic channel 1 does. However, when the guide wall protrudes toward the one side in the flow direction in the housing space from the top portion of the separation space wall, the first porous absorbing medium may protrude in the height direction closer to the top side than does the microfluidic channel.

A downstream portion 3a of the separation space 3 located downstream of the flow direction, is occupied by the first porous absorbing medium 2. The separation space 3 communicates with the microfluidic channel 1 and the two parallel ventilation passages 6 in the flow direction. Specifically, an upstream portion 3b of the separation space 3 located downstream of the flow direction, communicates with the microfluidic channel 1 and the two parallel ventilation passages 6 in the flow direction. Two ventilation spaces 3c are formed at the top portion 9a of the separation space wall 9.

The two ventilation spaces 3c respectively communicate with the two parallel ventilation passages 6 on the upstream side in the flow direction. The top portion 8a of the microfluidic channel wall 8, and the top portion 9a of the separation space wall 9, may each linearly extend continuously along the flow direction. The ventilation spaces 3c allow for air ventilation between the separation space 3 and the parallel ventilation passages 6. The two ventilation spaces 3c are located outside of the microfluidic channel 1 in the width direction. The distance between the two ventilation spaces 3c in the width direction may be substantially equal to the width of the microfluidic channel 1. The two ventilation spaces 3c may be respectively disposed corresponding to the two parallel ventilation passages 6 in the width direction. The two ventilation spaces 3c may communicate with the housing space 4. Specifically, the two ventilation spaces 3c may extend to communicate with the top portion of the housing space 4 in the height direction on the downstream side in the flow direction.

The volume Q of the separation space 3 may be in the range of about 0.001 μL to about 10000 μL, inclusive. The ratio of the volume Q of the separation space 3 to the volume P of the microfluidic channel 1, that is, Q/P may be greater than or equal to about 0.01. However, the volume of the separation space, and the ratio of the volume of the separation space to that of the microfluidic channel are not limited to these. The volume Q of the separation space 3 may be greater than the volume P of the microfluidic channel 1. However, the volume of the separation space may be set to be less than or equal to the volume of the microfluidic channel.

Furthermore, hydrophilization treatment may be applied to the respective surfaces of the microfluidic channel wall 8 and the separation space wall 9 that come in contact with a liquid. The hydrophilization treatment may be an optical treatment using plasma, or a treatment with a blocking agent capable of preventing a non-specific conjugate contained in a liquid, if any, from being adsorbed to those surfaces, or may include at least one of such treatments. Examples of the blocking agent include commercial blocking agents, such as Block Ace, bovine serum albumin, casein, skimmed milk, gelatin, surfactants, polyvinyl alcohol, globulin, serum (for example, fetal bovine serum or normal rabbit serum), ethanol, and MPC polymer. Such blocking agents may be used either alone or in combination by being mixed.

The inlet 5 is formed to penetrate through the top portion 8a of the microfluidic channel wall 8 in the height direction. Each of the parallel ventilation passages 6 is formed to be recessed to the top side and the bottom side of the microfluidic channel 1 in the height direction. The connecting ventilation passage 7 is formed to be recessed to the bottom side of the microfluidic channel 1 in the height direction. Atop portion 13a of the connecting ventilation passage wall 13 located on the top side in the height direction is disposed substantially corresponding to the top portion 8a of the microfluidic channel wall 8 in the height direction. The two parallel ventilation passages 6 and the connecting ventilation passage 7 may extend continuously to form a substantially U-like shape.

The guide wall 10 is disposed between the first porous absorbing medium 2 and a second porous absorbing medium 15 described below in the height direction. The guide wall 10 may be disposed to form a tapered shape toward the downstream side from the upstream side in the flow direction. However, the shape of the guide wall is not limited thereto.

The assay unit 100 includes the second porous absorbing medium 15 in addition to the first porous absorbing medium 2. The second porous absorbing medium 15 is located closer to the bottom side in the height direction than the first porous absorbing medium 2. However, if the guide wall protrudes toward the one side in the flow direction in the housing space from the top portion of the separation space wall, the second porous absorbing medium may be located closer to the top side in the height direction than the first porous absorbing medium. The first and second porous absorbing media 2 and 15 are in contact with each other in the height direction while having the guide wall 10 interposed therebetween. A liquid is fed to the second porous absorbing medium 15 via the first porous absorbing medium 2. The housing space 4 is configured to house the second porous absorbing medium 15 in addition to the first porous absorbing medium 2.

The assay unit 100 includes two ventilation passage vent holes 16 that respectively allow the two parallel ventilation passages 6 to communicate with the outside of the assay unit 100. Each ventilation passage vent hole 16 is formed to allow for air circulation from the outside of the assay unit 100 to each parallel ventilation passage 6 defined by one of the two parallel ventilation passage walls 12. Specifically, each ventilation passage vent hole 16 may be formed to penetrate through a top portion 12a of one of the two parallel ventilation passage walls 12 located on the top side in the height direction. In addition, the ventilation passage vent holes 16 are preferably provided above the first porous absorbing medium 2 in the vertical direction. However, the ventilation passage vent holes are not limited thereto. For example, only one ventilation passage vent hole may be provided on one of the two parallel ventilation passage walls. Alternatively, three or more ventilation passage vent holes may be provided.

The assay unit 100 includes a housing space vent hole 17 that allows the housing space 4 to communicate with the outside of the assay unit 100. The housing space vent hole 17 is formed to penetrate through the housing space wall 11. The housing space vent hole 17 may be located on the one side in the flow direction with respect to the housing space 4.

A fluidic channel top-side cavity 18 is formed on the top side of the top portion 8a of the microfluidic channel wall 8 in the height direction. A fluidic channel bottom-side cavity 19 is formed on the bottom side of the bottom portion 8b of the microfluidic channel wall 8 in the height direction. A separation space top-side cavity 20 is formed on the top side of the top portion 9a of the separation space wall 9 in the height direction. A housing space top-side cavity 21 is formed on the top side of a top portion 11a of the housing space wall 11 in the height direction.

One end of the fluidic channel top-side cavity 18, located on the one side in the flow direction, communicates with the separation space top-side cavity 20. The other end of the fluidic channel top-side cavity 18, located on the other side in the flow direction, is located at a distance from the inlet 5. The fluidic channel top-side cavity 18 communicates with the two parallel ventilation passages 6 in the width direction. The fluidic channel bottom-side cavity 19 is formed corresponding to the microfluidic channel 1 as seen in the height direction. The fluidic channel bottom-side cavity 19 communicates with the two parallel ventilation passages 6 in the width direction. The fluidic channel bottom-side cavity 19 also communicates with the connecting ventilation passage 7 in the flow direction. The separation space top-side cavity 20 is formed corresponding to the top portion 9a of the separation space wall 9 as seen in the height direction. The housing space top-side cavity 21 is disposed at a distance from the separation space top-side cavity 20 in the flow direction. The separation space top-side cavity 20 communicates with the two ventilation spaces 3c in the width direction. The housing space top-side cavity 21 communicates with the two ventilation spaces 3c in the height direction.

The assay unit includes the window portion 22 configured to allow a signal, which is derived from a substance that specifically binds to the detection section 14 in the microfluidic channel 1, to be visually observed from the outside of the assay unit. The window portion 22 may be configured to allow a signal to pass through the window portion, and is preferably transparent to light in the visible wavelength range. The window portion 22 is located on the top side of the fluidic channel top-side cavity 18 in the height direction. Furthermore, the window portion 22 may be located corresponding to the intermediate section 1c of the microfluidic channel 1, in particular, the detection section 14. The assay unit also includes a window portion 52 configured to allow a signal, which is derived from a substance that specifically binds to the internal standard section 54 in the microfluidic channel 1, to be visually observed from the outside of the assay unit. The window portion 52 can also be configured to allow a signal to pass therethrough, and is preferably transparent to light in the visible wavelength range, and is located on the top side of the fluidic channel top-side cavity 18 in the height direction. The window portion 52 may be located corresponding to the intermediate section 1c of the microfluidic channel 1, in particular, the internal standard section 54. In the embodiment shown in the drawings, the detection section 14 is provided on the upstream side close to the inlet 5, and the internal standard section 54 is provided on the downstream side close to the porous medium 2. However, it is also possible to provide the internal standard section 54 on the upstream side close to the inlet 5, and provide the detection section 14 on the downstream side close to the porous medium 2. In such a case, the window portions 22 and 52 corresponding to the respecting sections may be provided.

### Layered Structure of Assay Unit 100

The layered structure of the assay unit 100 will be described with reference to FIG. 2. That is, the assay unit 100 according to the present embodiment may be produced using the following layered structure, for example. Obviously, the assay unit 100 may also be produced using a structure other than such a layered structure.

The assay unit 100 includes a top-side casing layer S1, a top-side cavity layer S2, a top-side core layer S3, an intermediate core layer S4, a bottom-side core layer S5, a bottom-side cavity layer S6, an intermediate spacer layer S7, an intermediate adhesion layer S8, a bottom-side spacer layer S9, a bottom-side adhesion layer S10, and a bottom-side casing layer S11 that are arranged in this order from the top to bottom of the assay unit 100. Each of the top-side casing layer S1, the top-side core layer S3, the bottom-side core layer S5, the intermediate spacer layer S7, the bottom-side spacer layer S9, and the bottom-side casing layer S11 is formed using a material that does not allow a liquid to pass therethrough. The contact angle of the material of the top-side core layer S3 and the bottom-side core layer S5 may be less than 90°. The top-side core layer S3 and the bottom-side core layer S5 may be transparent. However, at least one of the top-side core layer and the bottom-side core layer may be translucent or opaque. At least one of the top-side core layer S3 and the bottom-side core layer S5 is elastically deformable under liquid pressure when a liquid is passed through the assay unit 100.

Furthermore, each of the top-side casing layer S1, the top-side core layer S3, the bottom-side core layer S5, the intermediate spacer layer S7, the bottom-side spacer layer S9, and the bottom-side casing layer S11 may be manufactured of plastic. Furthermore, a material for forming each of the top-side casing layer S1, the top-side core layer S3, the bottom-side core layer S5, the intermediate spacer layer S7, the bottom-side spacer layer S9, and the bottom-side casing layer S11 may be a plastic sheet or film. Examples of such plastic include polyolefins (PO), such as polyethylene (PE), high density polyethylene (HDPE), and polypropylene (PP); ABS resins (ABS); AS resins (SAN); polyvinylidene chloride (PVDC); polystyrene (PS); polyethylene terephthalate (PET); polyvinyl chloride (PVC); nylon; polymethyl methacrylate (PMMA); cycloolefin copolymer (COC); cycloolefin polymer (COP); polycarbonate (PC); polydimethylsiloxane (PDMS); polyacrylonitrile (PAN); biodegradable plastic, such as polylactic acid (PLA); other polymers; and combinations thereof. However, when at least one of the top-side casing layer, the top-side core layer, the bottom-side core layer, the spacer layer, and the bottom-side casing layer is formed of a material that does not allow for fluid infiltration, it is possible to produce the layer using a material other than plastic. The material other than plastic may be resin, glass, or metal, for example. The materials or ingredients used for forming the top-side casing layer S1, the top-side core layer S3, the bottom-side core layer S5, the intermediate spacer layer S7, the bottom-side spacer layer S9, and the bottom-side casing layer S11 may be either the same or different.

Each of the top-side cavity layer S2, the intermediate core layer S4, the bottom-side cavity layer S6, the intermediate adhesion layer S8, and the bottom-side adhesion layer S10 is in the form of a double-sided adhesive tape or a layer including the double-sided adhesive tape. The top and bottom surfaces of such layers S2, S4, S6, S8, and S10 exhibit adhesive properties. The top and bottom surfaces of the top-side cavity layer S2 are respectively bonded to the bottom surface of the top-side casing layer S1 and the top surface of the top-side core layer S3. The top and bottom surfaces of the intermediate core layer S4 are respectively bonded to the bottom surface of the top-side core layer S3 and the top surface of the bottom-side core layer S5. The top and bottom surfaces of the bottom-side cavity layer S6 are respectively bonded to the bottom surface of the bottom-side core layer S5 and the top surface of the intermediate spacer layer S7. The top and bottom surfaces of the intermediate adhesion layer S8 are respectively bonded to the bottom surface of the intermediate spacer layer S7 and the top surface of the bottom-side spacer layer S9. The top and bottom surfaces of the bottom-side adhesion layer S10 are respectively bonded to the bottom surface of the bottom-side spacer layer S9 and the top surface of the bottom-side casing layer S11.

However, at least one of the top-side cavity layer, the intermediate core layer, the bottom-side cavity layer, the intermediate adhesion layer, and the bottom-side adhesion layer may be produced using the materials or ingredients that may be used for forming the top-side casing layer, the top-side core layer, the bottom-side core layer, the intermediate spacer layer, the bottom-side spacer layer, and the bottom-side casing layer as described above. In such a case, the adjacent layers may be bonded together using a bonding means, such as an adhesive, or by welding. The materials or ingredients used for forming at least one of the top-side cavity layer, the intermediate core layer, the bottom-side cavity layer, the intermediate adhesion layer, and the bottom-side adhesion layer may be either the same as or different from those used for forming the adjacent layer.

### Relationship Between Components and Layered Structure of Assay Unit

Referring to FIG. 2 and FIGS. 4 to 6, description will be made of, regarding a case in which the assay unit 100 according to the present embodiment is produced using the foregoing layered structure, the relationship between the components and the layered structure of the assay unit 100. The microfluidic channel 1 is formed to penetrate through the intermediate core layer S4 in the height direction. The top portion 8a and the bottom portion 8b of the microfluidic channel wall 8 are respectively formed in the top-side core layer S3 and the bottom side core layer S5.

The separation space 3 is formed to penetrate through the intermediate core layer S4 in the height direction. The ventilation spaces 3c are formed to penetrate through the top-side core layer S3 in the height direction. The top portion 9a of the separation space wall 9 is formed in the top-side core layer S3. The bottom portion 9b of the separation space wall 9 is formed in the bottom-side core layer S5. The housing space 4 is formed to include seven through sections 4a, 4b, 4c, 4d, 4e, 4f, and 4g that respectively penetrate through the intermediate core layer S4, the bottom-side core layer S5, the bottom-side cavity layer S6, the intermediate spacer layer S7, the intermediate adhesion layer S8, the bottom-side spacer layer S9, and the bottom-side adhesion layer S10 in the height direction. The top portion 11a and the bottom portion 11b of the housing space wall 11 are respectively formed in the top-side core layer S3 and the bottom-side casing layer S11.

Among the seven through sections 4a to 4g forming the housing space 4, the four top-side through sections 4a to 4d are formed to allow the first porous absorbing medium 2 to be housed therein. The three bottom-side through sections 4e to 4g are formed to allow the second porous absorbing medium 15 to be housed therein. The second porous absorbing medium 15 may be larger than the first porous absorbing medium 2. In particular, the length of the second porous absorbing medium 15 in the flow direction may be greater than that of the first porous absorbing medium 2.

The inlet 5 is formed to include three through sections 5a, 5b, and 5c that respectively penetrate through the top-side casing layer S1, the top-side cavity layer S2, and the top-side core layer S3 in the height direction. The guide wall 10 is formed in the bottom-side core layer S5. Each parallel ventilation passage 6 is formed to include five through sections 6a, 6b, 6c, 6d, and 6e that respectively penetrate through the top-side cavity layer S2, the top-side core layer S3, the intermediate core layer S4, the bottom-side core layer S5, and the bottom-side cavity layer S6 in the height direction. The top portion 12a and the bottom portion 12b of the parallel ventilation passage wall 12 are respectively formed in the top-side casing layer S1 and the intermediate spacer layer S7. The connecting ventilation passage 7 is formed to include two through sections 7a and 7b that respectively penetrate through the bottom-side core layer S5 and the bottom-side cavity layer S6 in the height direction. The top portion 13a and the bottom portion 13b of the connecting ventilation passage wall 13 are respectively formed in the intermediate core layer S4 and the intermediate spacer layer S7.

Two ventilation passage vent holes 16 are formed in a single assay unit. The respective ventilation passage vent holes 16 are formed to penetrate through the top-side casing layer S1 in the height direction, and to allow the parallel ventilation passages 6 to communicate with the outside of the assay unit 100. The ventilation passage vent holes 16 are located above the first porous absorbing medium 2 in the vertical direction, and also function as ventilation passages for promoting the evaporation of liquid absorbed by the porous absorbing medium 2. The housing space vent hole 17 is formed to penetrate through the bottom-side casing layer S11 in the height direction, and to allow the housing space 4 to communicate with the outside of the assay unit 100.

The fluidic channel top-side cavity 18, the separation space top-side cavity 20, and the housing space top-side cavity 21 are formed to penetrate through the top-side cavity layer S2 in the height direction. The fluidic channel top-side cavity 18, the separation space top-side cavity 20, and the housing space top-side cavity 21 are located between the top-side casing layer S1 and the top-side core layer S3 in the height direction. The fluidic channel bottom-side cavity 19 is formed to penetrate through the bottom-side cavity layer S6 in the height direction. The fluidic channel bottom-side cavity 19 is located between the bottom-side core layer S5 and the intermediate spacer layer S7 in the height direction. The window portion 22 is formed in the top-side casing layer S1.

The assay device of the present invention includes two or more assay units 100 described above. In the assay device, the plurality of assay units 100 are preferably disposed so that their microfluidic channels 1 are parallel with each other. Furthermore, in the adjacent assay units, the inlets 5, the detection window portions 22, and the internal standard window portions 52 are preferably disposed linearly. The number of assay units included in the assay device is not limited to a specific number, and may be 2, 3, 4, 5, 6, 7, 8, or more. Any number of assay units may be included in a single assay device so as to be compatible with a device for reading signals from the detection section 14 and the internal standard section 54 in the assay method described below.

The plurality of assay units 100 included in the assay device preferably have the same structure. However, for example, the plurality of assay units 100 included in a single assay device may have different structures. For example, the detection antibody immobilized on the detection section 14 and the internal standard substance immobilized on the internal standard section 54 in a given microfluidic channel 1 may be different from those in the other microfluidic channels.

In the production of the assay device, it is preferable to form the detection section 14 and the internal standard section 54 in predetermined regions of the bottom portion 8b of the microfluidic channel wall 8, which forms the bottom-side core layer S5, before stacking the layers S1 to S11 and bonding them together. The detection section 14 is formed by directly immobilizing a predetermined amount of a substance configured to be able to specifically react with the target substance (that is, the detection antibody) on the bottom portion 8b or the top portion 8a, or impregnating the reaction porous medium disposed on the bottom portion with the substance. The internal standard section 54 is also formed by directly immobilizing a predetermined amount of an internal standard substance on the bottom portion 8b or the top portion 8a, or impregnating the reaction porous medium disposed on the bottom portion with the internal standard substance. Then, blocking and drying may be performed. Although FIG. 2 schematically illustrates a stack of a single assay unit, it is possible to produce an assay device including a plurality of assay units by forming, in each of the layers S1 to S11, cavities and through sections in rows corresponding to the plurality of assay units, and stacking such layers. Alternatively, as in FIG. 2, it is also possible to arrange the assay units in the width direction, and connect the first and second porous absorbing media in the width direction.

The assay device according to the first embodiment is not limited to the foregoing aspect, and may have other structures included in Patent Document 1 (WO 2020/045551) by the same inventor. For example, the assay device may be configured such that when a liquid is supplied to the microfluidic channel from the inlet, at an end of the microfluidic channel close to the inlet, an abutment state of the top portion and the bottom portion of the micro microfluidic channel wall in the height direction is made changeable into a state in which those portions are separated in the height direction. In the assay device, the microfluidic channel may be formed to have its width decreased toward one side from the end of the microfluidic channel close to the inlet. With the structure disclosed in Patent Document 1, the accuracy of a liquid flow in the microfluidic channel can be further increased. Therefore, reactions that occur in the detection section have no influence on reactions that occur in the internal standard section, and thus, the accuracy of reactions that occur in the detection section can be ensured with the internal standard section.

### Second Embodiment: Assay Method

The second embodiment of the present invention relates to an assay method. The assay method uses the foregoing assay device, and includes the following steps, performed in sequence:
(a) applying a sample to each microfluidic channel,
(b) applying a cleaning solution to the microfluidic channel, and
(c) applying a liquid containing a first label capable of specifically binding to a target substance, and a second label capable of specifically binding to the internal standard substance, to the microfluidic channel.

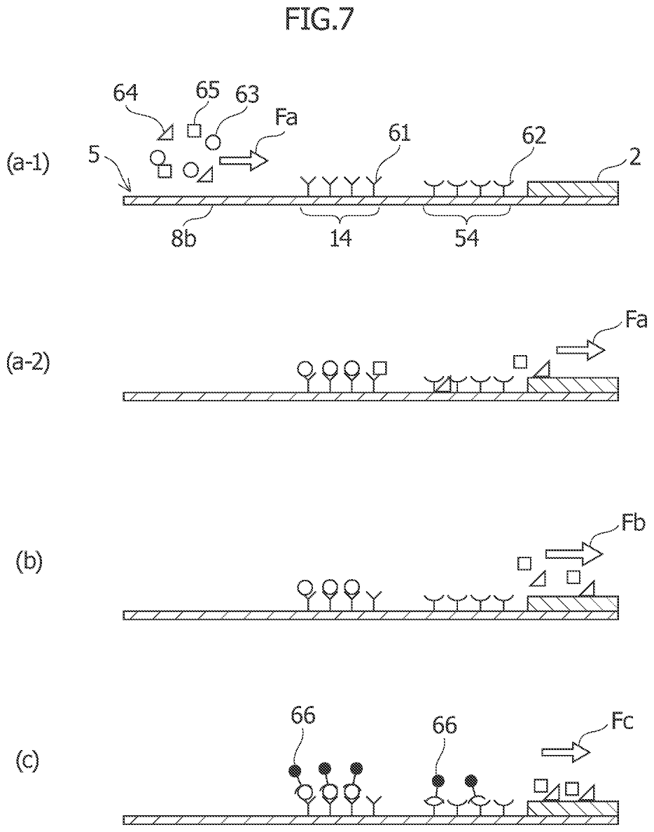

The assay method according to the present invention will be specifically described with reference to FIG. 7. FIG. 7 illustrates an example of an assay method intended to detect an antigen “A” that is one type of target substance. However, the present invention is not limited to the detection of such a specific antigen A. Furthermore, the present invention can be used not only for the detection of one type of target substance, but also for the detection of two or more types of target substances. FIG. 7 schematically shows the time-series movement of substances in the microfluidic channel 1 of the single assay unit 100 in the assay device of FIGS. 2 to 6 when the assay method of the present embodiment is performed. Referring to FIG. 7(a−1), the left end corresponds to the inlet 5, and the right end corresponds to the first porous absorbing medium 2. The microfluidic channel is provided with the detection section 14 on the upstream side in the flow direction of a liquid, and is provided with the internal standard section 54 on the downstream side. The detection section 14 is a region including an antibody 61 immobilized on the bottom portion 8b of the microfluidic channel. The antibody 61 is the detection antibody that specifically recognizes the antigen A. The internal standard section 54 is a region including an antibody 62 that is an internal standard substance immobilized on the bottom portion 8b of the microfluidic channel. The antibody 62 is an antibody that does not recognize the antigen Abut specifically recognizes a labeled antibody 66.

### Step of Performing Operation Using Assay Device

In the step (a), a sample is applied to the inlet 5 of the microfluidic channel. The application may be performed by dropping one or more droplets of the sample (about several tens of μL/droplet) on the inlet 5. The dropping may be performed once or be repeated more than once. The amount of the sample necessary for the assay method is applied. When the dropping is repeated more than once, a plurality of droplets may be continuously dropped. Alternatively, the intervals of the timing of dropping may be set to given time intervals, such as every three minutes or every five minutes, for example. The sample is a liquid containing an antigen A63, which is a target substance for an assay, and other antigens 64 and 65. FIG. 7(a−1) shows a state in which the sample is applied to the inlet 5, and a flow of the liquid is indicated by an arrow Fa.

The antigens 63, 64, and 65 flow through the microfluidic channel as the liquid moves based on the lateral flow. FIG. 7(a−2) shows a state in which the liquid has flowed through the microfluidic channel and reached the first porous absorbing medium 2. The antigen A63 in the sample specifically reacts with and binds to the antibody 61, and some of the antigens 64 and 65 remain in the microfluidic channel due to non-specific interaction or physical action, for example, while others are carried to the first porous absorbing medium 2 together with the liquid.

The one or more other assay units included in the assay device can also perform the step (a) sequentially. Although it is not specifically mentioned, the following steps are also preferably performed sequentially by the plurality of assay units in a similar way.

Then, in the step (b), a cleaning solution is applied to the microfluidic channel. The cleaning solution may be any solution that has no influence on the specifically bound state of the antigen A63 and the antibody 61, and can remove the antigens residing in the microfluidic channel. As the cleaning solution, a surfactant may be used, for example, but the cleaning solution is not limited to a specific solution. The application of the cleaning solution may be performed by dropping one or more droplets of the cleaning solution (about several tens of μL/droplet) on the inlet 5. The dropping may be performed once or may be repeated more than once. When the dropping is repeated more than once, droplets may be continuously dropped. The continuous addition of the cleaning solution is advantageous in that it can completely clean up non-immobilized substances in the microfluidic channel. Alternatively, droplets may be sequentially dropped at given time intervals. A flow of the cleaning solution is indicated by arrow Fb. FIG. 7(b) shows the state of the fluidic channel that has been cleaned. The antigens 64 and 65 present in the fluidic channel in FIG. 7(a−2) have been removed.

Then, in the step (c), a liquid containing a first label capable of specifically binding to the target substance, and a second label capable of specifically binding to the internal standard substance, is applied to the microfluidic channel. In the present aspect, the first label capable of specifically binding to the antigen A63 as the target substance, and the second label capable of specifically binding to the antibody 62 as the internal standard substance, are the same, which are the labeled antibodies 66. Thus, in the present step, a liquid containing the labeled antibodies 66 is applied to the microfluidic channel. The application of the labeled antibodies 66 may also be performed by dropping droplets (about several tens of μL/droplet) on the inlet 5. The dropping may be performed once or may be repeated more than once. The amount of the labeled antibodies 66 needed to obtain desired signals is preferably dropped appropriately. The concentration of the liquid containing the labeled antibodies 66 may be set to a concentration that is sufficient for the labeled antibodies 66 to be present while being able to bind to the antigens A63, which have bound to the antibodies 61, and the antibodies 62 as the internal standard substance when the liquid is applied to the microfluidic channel 1 in the step (c). While the liquid containing the labeled antibodies 66 flows through the microfluidic channel 1, some of the labeled antibodies 66 specifically react with and bind to the antigens A63 or the antibodies 62. The labeled antibodies 66 that have not been bound flow toward the first porous absorbing medium 2 together with the liquid. The flow of the liquid is indicated by arrow Fc. FIG. 7(c) shows the state of the fluidic channel after the completion of the step (c).

In another aspect (not shown in the drawings), when the internal standard substance is an antigen, the label may be a molecule including an antibody portion to bind to the antigen, and one skilled in the art may freely select the label corresponding to the type of the internal standard substance. In addition, the first label and the second label may be different types of substances. An aspect of the first label and the second label that are of different types will be described below.

After the completion of the step (c), a step of applying a cleaning solution may be further provided as appropriate. This is to completely remove the unreacted labeled antibodies 66 from the microfluidic channel. Through the steps (a) to (c), both the detection section 14 and the internal standard section 54 become able to detect signals of the labeled antibodies 66.

When the labeled antibody 66 is a substance that generates a detectable signal, a step of measuring signals described below (a step (d)) may be performed after the completion of the step (c). If the labeled antibody 66 is not a substance that generates a detectable signal, it is possible to further provide a step of applying a substance to the microfluidic channel, which substance imparts a signal-generating capability to the labeled antibody 66. The detectable signal may be visible light, fluorescence, chemiluminescence, electrochemistry, electrochemiluminescence, or plasmon, for example. The substance that provides signal generating capability may be a substance corresponding to such signals, for example, a color former. Examples of the portion of the label involved in generating a signal include, but are not limited to, AP (alkaline phosphatase), rhodamine, biotin, FITC (fluorescein), PE (phycoerythrin), Cy (cyanine), APC (allophycocyanin), Alexa, and DyLight in addition to HRP exemplarily described above. Examples of a substrate for HRP in colorimetric ELISA include TMB (3,3′,5,5′-tetramethylbenzidine), OPD (o-phenylenediamine dihydrochloride), and ABTS (2,2′-azinobis-(3-ethylbenzothiazoline-6-sulfonic acid)-diammonium salt). Examples of a substrate for AP include, but are not limited to, PNPP (p-nitrophenyl phosphate, disodium salt).

### Measurement and Quantification of Signals

After the steps (a) to (c), the assay method according to the present embodiment may further include the following step:
(d) measuring a signal of the first label in the detection section and a signal of the second label in the internal standard section.

The method of measuring a signal differs depending on the type of the signal. One of ordinary skill in the art may quantify a predetermined signal by performing a measurement method suitable for the signal. For example, to measure a visible light signal from each of the detection section 14 and the internal standard section 54, the absorbance or brightness of each section may be measured. Detectable signals other than visible light signals may also be appropriately measured using conventional publicly known methods.

The measurement of absorbance, which is an example of a method of quantifying a visible light signal, can be performed using a spectrophotometer. The measurement wavelength may be appropriately determined based on the absorption wavelength of a substance that generates a signal. For example, to quantify a blue light signal, absorption of light with a wavelength of 652 nm may be measured. Alternatively, it is also possible to capture a digital collar image of each of the detection section 14 and the internal standard section 54, and obtain absorbance based on the brightness of the image. More specifically, digital color images of the measurement point, such as the detection section 14 or the internal standard section 54, and a reference point are captured with a smartphone or a digital camera. The reference point may be a white part, such as the housing of the assay device, or a color sample, for example. Next, each of the obtained digital color images is decomposed into the three primary colors of red (R), green (g), and blue (b) so that the brightness of each component is obtained. “Brightness” refers to, when a target color is expressed with a mixed color of red (wavelength: 700.0 nm), green (wavelength: 546.1 nm), and blue (wavelength: 435.8 nm) based on the CIE 1931 color space defined by the International Commission on Illumination, the magnitude of the R, G, or B component. For example, to quantify a blue light signal, it is possible to obtain absorbance based on the following equation by obtaining the brightness of the red (R) component of each of the measurement point and the white part.

Absorbance=−log(the brightness of R of the measurement point/the brightness of R of the white part)

To quantify a signal other than a blue light signal, it is similarly possible to quantify absorbance based on one of R, G, and B, or a combination of two or more of them according to the absorption wavelength of a substance that generates the signal.

The measured value of the signal of the first label in the detection section obtained in the step (d) can be used as it is or through an appropriate calculation for the quantification or semi-quantitation of the target substance. Examples of the appropriate calculation include a method of converting the measured value of the signal into a concentration based on a master calibration curve obtained in advance. The master calibration curve can be obtained from a sample containing a known concentration of the target substance.

When the calculation based on the master calibration curve is performed, it is also possible to obtain a deviation rate with respect to the master calibration curve, and also to perform a calculation for correcting the master calibration curve. Correction of the master calibration curve is performed by flowing a positive control, which is a sample containing a known concentration of the target substance, through the microfluidic channel of each assay device. For example, when the absorbance of a signal is measured using the assay device shown in FIG. 1, in the step (a), a positive control is applied to the microfluidic channel of at least one assay unit, while a sample as a measurement target is flowed through the microfluidic channels of the other assay units. In the step (d), absorbance obtained from the detection section 14 of the assay unit to which the positive control has been applied is divided by absorbance in the master calibration curve at the corresponding concertation of the target substance, whereby a deviation rate with respect to the master calibration curve can be obtained. To obtain an accurate deviation rate, it is preferable to use two types of positive controls including a high-concentration positive control and a low-concentration positive control, and obtain deviation rates for the two concentrations, and then determine the mean value thereof as a deviation rate (a deviation constant) specific to the assay device. Then, for a sample containing an unknown concentration of the target substance as the measurement target, correction is performed through a calculation, for example, multiplying the absorbance obtained in the step (d) by the deviation constant or an inverse of the deviation constant. Then, the corrected absorbance is applied to the master calibration curve so that the concentration of the target substance in each sample can be obtained.

Ensuring of Reaction Based on Signal from Internal Standard Section

The assay method according to the present embodiment may include the following step in an aspect:
(g) a step of determining if each assay unit is defective based on the signal of the second label obtained in the step (d).

The measured signal of the second label in the internal standard section obtained in the step (d) can be used to ensure a reaction that has occurred in the detection section 14. That is, when the signal of the second label is less than or equal to a specific threshold, or greater than or equal to the threshold, it is possible to determine that a reaction that has occurred in the detection section is abnormal. The threshold depends on the concentration of the internal standard substance, the portion on which the internal standard substance is immobilized (that is, the top portion or the bottom portion of the fluidic channel, or both), the thickness of the microfluidic channel (the distance between S3 and S5), the type of the target substance (considering cross-reactivity), and the type of the blocking agent. The threshold also depends on a reaction protocol, for example, a reaction time, and the type and the concentration of the cleaning solution. Thus, one skilled in the art may determine the threshold by performing a preliminary experiment, considering the structure of the assay device and the reaction protocol. If the steps (a) to (c) proceed normally, it is in principle possible to obtain a constant signal from the second label regardless of the amount of the target substance present. However, when the signal of the second label is less than or equal to the threshold, or is greater than or equal to the threshold, it is suspected that the assay unit is defective. Specifically, when the signal of the second label is less than or equal to the threshold, it is suspected that the device has an abnormality, and when the signal of the second label is greater than or equal to the threshold, it is considered that an abnormal color has been exhibited perhaps because the label has not been cleaned sufficiently.

The threshold may be determined by creating a calibration curve that shows the relationship between the concentration of the second label and signals. The calibration curve may differ depending on the type of the second label used, the type and the concentration of the internal standard substance immobilized on the assay device, and the thickness of the microfluidic channel of the assay device, for example. Thus, creating a calibration curve for each case can determine the threshold. The determination of the threshold based on such a method is particularly effective when the second label includes an HRP antibody and TMB, for example.

Correction of Signal from Detection Section Based on Signal from Internal Standard Section

When the first label and the second label applied to the microfluidic channel in the step (c) are different, the following steps of performing calculations may be further provided after the step (d) in an aspect:
(e) a step of determining, based on a calibration curve of the internal standard substance obtained in advance and the signal of the second label obtained in the step (d), a deviation rate with respect to the calibration curve of the internal standard substance; and
(f) a step of obtaining, based on the deviation rate, the signal of the first label obtained in the step (d), and a calibration curve of the target substance obtained in advance, a corrected concentration of the target substance.

The present aspect can be performed when the first label and the second label applied to the microfluidic channel in the step (c) are different. Examples of such a case include, but are not particularly limited to, a case in which the target substance is an allergen, the internal standard substance is an antibody produced from specific animal species, the first label is an RP-labeled anti-allergen antibody (that is, an antibody produced from different animal species from the internal standard substance), and the second label is an RP-labeled animal antibody (that is, an antibody produced from the same animal species as the internal standard substance). For example, the first label may be an RP-labeled anti-allergen antibody produced from an animal other than a rabbit, the internal standard substance may be an anti-rabbit IgG antibody, and the second label may be an RP-labeled rabbit antibody. When the calculations in the steps (e) and (f) are performed after the step (d), it is possible to correct the master calibration curve of the target substance based on the signal from the internal standard section without flowing the foregoing positive control through the microfluidic channel.

Specifically, the master calibration curve of the target substance is created for a known concentration of the target substance in advance. In addition, the master calibration curve of the internal standard substance is created by changing the concentration of the second label. Next, the steps (a) to (d) are performed. For example, to measure the absorbance of a signal using the assay device shown in FIG. 1, it is possible to apply a measurement target sample to each of the microfluidic channels of six assay units without using a positive control in the step (a). In the step (e) of the present aspect, the absorbance of the internal standard section 54 of each assay unit obtained in the step (d) is divided by the absorbance of the internal standard substance in the master calibration curve of the internal standard substance at the corresponding concentration (immobilized amount) so that a deviation rate with respect to the master calibration curve of the internal standard substance can be obtained. In the following step (f), the absorbance of the detection section 54 of each assay unit obtained in the step (d) is multiplied by the deviation rate obtained in the step (e) so that the corrected absorbance of the detection section 54 is obtained. Finally, the corrected absorbance of the detection section 54 is applied to the master calibration curve of the target substance so that the concentration of the target substance in each sample can be obtained.

According to the present aspect, since a deviation rate can be obtained for each microfluidic channel, there is no need to use a positive control. Therefore, all of the microfluidic channels in a single assay device can be used for measuring a sample, and the concentration of the target substance can be corrected based on the internal standard substance, enabling simpler and more accurate precision control.

### Visual Detection of Signal

According to an embodiment of the present invention, a user is able to visually check the signal obtained in the step (c). Such an aspect of detection is possible when it is intended to perform a simple assay for detecting the presence or absence of allergenic substances in food, for example. In the present aspect, whether a sample contains a target substance is determined mainly based on whether there is a signal from the detection section 14. Furthermore, regardless of whether there is a signal from the detection section 14, checking a signal from the internal standard section 54 can ensure that an assay reaction in the detection section 14 has proceeded normally. Furthermore, if the assay device is made to include a color sample, and a signal from the detection section 14 is compared with the color sample, visual semi-quantification of the signal becomes possible. The color sample may be provided on the top portion of the assay device by printing, or may be provided integrally with the assay device, or alternatively, the color sample may be provided separately from the body of the assay device.

### Detection of Plurality of Target Substances

If the assay device according to the first embodiment includes two or more detection sections, it is possible to simultaneously detect a plurality of target substances. More specifically, it is possible to, by providing a plurality of detection sections and their corresponding window portions, produce an assay device in which the plurality of detection sections respectively have immobilized thereon different detection antibodies that can respectively specifically bind to a plurality of target substances. In the assay method, a liquid containing labels, which can each specifically bind to a plurality of target substances, can be applied to each microfluidic channel in the step (c). For example, to detect two different types of target substances, it is possible to provide, instead of the step (c), a step (c′) of applying a liquid containing a first label capable of specifically binding to a first target substance; a second label capable of specifically binding to a substance immobilized on the internal standard section; and a third label capable of specifically binding to a second target substance, to each microfluidic channel. Alternatively, it is also possible to apply first to third labels that can generate different fluorescence signals to a plurality of different detection antibodies. By such operations, multiple target substances can be simultaneously measured.

### Detection of Saturation

According to an embodiment of the present invention, the step (d) may further include a step of measuring a signal of the first label from the detection section over time, and a step of determining if the detection has failed based on changes in the signal over time.

Depending on a substance that generates a signal, an undesirable change in the signal, which would hinder the accurate quantification of a target substance, may occur in a region in which the concentration of the target substance is high. When the measurement of the signal in the step (d) is performed multiple times, over time, starting from a time point when a substance that generates a signal (for example, a color former) is added, it is possible to determine that the detection has failed if an undesirable change in the signal is obtained.

For example, regarding TMB, which is a preferably used color former, a visually observable signal sequentially changes from white to blue and yellow with an increase in the concentration of a target substance in a sample. When the signal changes from white to blue, absorbance increases, and the brightness of the red (R) component decreases. When the signal changes from blue to yellow, absorbance decreases, and the brightness of R increases. In the assay method of the present invention, the measurement in the step (d) is performed using TMB, and to quantify a target substance, it is preferable to use a change in the absorbance or the brightness of R in a region in which a signal changes from white to blue. However, depending on the concentration of the target substance, a reaction involving a change in a signal from blue to yellow may occur. Such a reaction can be perceived as an undesirable signal, that is, a signal indicating supersaturation. When such a change in the signal is obtained, it is possible to determine that the detection has failed, and thus terminate the reaction.

As a more specific example, the absorbance or the brightness of R of the detection section is measured at predetermined intervals starting from a time point when TMB is dropped, for example. The predetermined intervals may differ depending on the type of the color former used, but may be about 10 seconds to 1 minute, for example. The measurement is preferably continued until 10 to 20 minutes have elapsed from the time point of dropping TMB. Then, if the absorbance of the detection section has decreased two times in succession, or if the brightness of R of the detection section has increased two times in succession during the measurement over time, for example, such a phenomenon can be perceived as an “undesirable change in the signal,” and thus, it is possible to determine that the detection has failed. Examples of a substance that undergoes a change in the exhibited color as with TMB include o-phenylenediamine (OPD).

Providing such a step for detecting saturation can prevent erroneous detection for, in particular, a sample containing a high concentration of a target substance, and thus can accurately quantify the target substance.

According to the assay method of the second embodiment of the present invention, it is possible to obtain a signal based on the internal standard section in addition to a signal based on a reaction of a target substance in the detection section, and thus ensure the accuracy of the reaction of the target substance in the detection section.

### Third Embodiment: Method of Selecting Assay Device Using Evaluation Reagent

A method of selecting an assay device, that is, a method of evaluating the accuracy of the production of the assay device can be performed by flowing an evaluation reagent through the microfluidic channels of the assay device of the present invention. Specifically, after the completion of an assay, an evaluation reagent solution is applied to each of the microfluidic channels of a plurality of assay units of the assay device, and a signal from the detection section 14 and a signal from the internal standard section 54 are measured so that the thickness of each microfluidic channel can be obtained. Examples of the signal include, but are not limited to, absorbance and brightness. Hereinafter, description will be made of an example in which absorbance is used as the signal.

As the evaluation reagent, any reagent that can quantify a liquid in the microfluidic channel based on absorbance, brightness, or other signals may be used. Specific examples of the reagent include, but are not limited to, methylene blue and a copper sulfate aqueous solution. Droplets of such an evaluation reagent may be dropped (about several tens of μL/droplet) to be applied to each of the plurality of microfluidic channels 1 included in the assay device.

Next, the absorbance of the evaluation reagent in the detection section 14, and the absorbance of the evaluation reagent in the internal standard section 54 are measured. Such operations are performed on two or more assay devices, preferably, about five assay devices to calculate a coefficient of variation (CV). If the coefficient of variation (CV) is within a predetermined numerical range, for example, less than or equal to about 5%, it is possible to confirm that variation among the properties of the plurality of assay units included in the assay device is within a proper range, that is, the accuracy of the production of the assay device is greater than or equal to a predetermined criterion. Then, a non-defective assay device may be selected based on the result. That is, it is possible to confirm whether the liquid has flowed through each microfluidic channel, and if the thickness of each microfluidic channel (the distance between S3 and S5 in FIG. 2) is within a given range in the detection section 14 and in the internal standard section 54. The thickness of each microfluidic channel can be obtained from a calibration curve created in advance. More specifically, for example, the thickness of each microfluidic channel is measured using a three-dimensional shape measurement device, and a calibration curve is created for the absorbance of the evaluation reagent with respect to the thickness of the microfluidic channel so that the thickness of the microfluidic channel can be obtained based on the calibration curve.

In the selection method according to the present embodiment, it is also possible to select a non-defective assay device by flowing the evaluation reagent solution through all of the microfluidic channels after an assay has been performed, and individually evaluating accuracy for the respective microfluidic channels. Such an evaluation method is advantageous when it is considered that each assay unit (or each microfluidic channel) has a different property. Alternatively, when it can be regarded that assay devices produced in the same lot have no variation, it is possible to, by selecting one of the assay devices produced in the same lot and flowing the evaluation reagent therethrough for checking purposes, evaluate if given accuracy is ensured for all of the assay devices produced in the same lot and the assay units (or the microfluidic channels) included therein.

### Examples

Hereinafter, the present invention will be described more specifically by way of example. However, the following examples are not intended to limit the present invention.

### (1) Production of Assay Device

An assay device including six assay units shown in FIG. 1 was produced, and operation check for performing an assay method was performed on the assay device. To produce the assay device, 20 μL of 5.175 μg/mL anti-allergen antibodies (antibodies to wheat gliadin) were immobilized as the detection antibodies on the detection section 14 in the bottom-side core layer S5 forming the bottom portion 8b of the microfluidic channel wall 8, and 20 μL of 5 μg/mL anti-mouse IgG antibodies was immobilized as the internal standard substance on the internal standard section 54. Regarding the top-side casing layer S1 of the assay device, a white ABS resin was used for a portion that is visible from the outside of the device. Then, as shown in FIG. 2, the layers S1 to S11 as well as the other members to form the assay device were bonded together so that the assay device was produced. The production of the device and the following experimental operations were all performed at room temperature.

(2) Checking of Signal from Internal Standard Section

Four droplets of a cleaning solution (a phosphate buffer solution containing a surfactant) were dropped (about 40 μL/droplet) onto each microfluidic channel of the produced assay device so that the microfluidic channel was cleaned. After the cleaning, two droplets of a blocking agent (a phosphate buffer solution containing protective protein and glucose) were applied (about 20 μL/droplet) to each microfluidic channel so that blocking was performed for 60 minutes. Next, aspirating the blocking agent and drying the microfluidic channel were performed at room temperature for 30 minutes. Then, five droplets of a cleaning solution were applied (about 40 μL/droplet) to the microfluidic channel to be cleaned.

A liquid containing horseradish peroxidase (tRP)-labeled antibodies was applied as the labeled antibodies to the six assay units. In the assay device shown in FIG. 1, the fluidic channels (or the assay units) are sequentially numbered as fluidic channels 1 to 6 from the left side. Two droplets of 0 μg/mL, 0.03125 μg/mL, 0.0625 μg/mL, 0.125 μg/mL, 0.25 μg/mL, and 0.5 μg/mL RP-labeled antibodies were respectively applied to the fluidic channels 1 to 6 (40 μL/droplet). After 5 minutes had elapsed, eight droplets of a cleaning solution were applied (about 40 μL/droplet) to each microfluidic channel to be cleaned. Finally, a droplet of about 40 μL of a color former (tetramethylbenzidine; TMB) was applied.

The absorbance of the internal standard section 54 in each of the fluidic channels 1 to 6 was obtained, before the addition of the color former (TMB 0 min), after 5 minutes had elapsed from the addition (TMB 5 min), and after 10 minutes had elapsed from the addition (TMB 10 min). In the present example, absorbance was obtained using the following equation.

Absorbance=−log(the brightness of R of the measurement portion/the brightness of R of the white portion)

The measurement portion herein corresponds to the detection section or the internal standard section, and the white portion herein corresponds to the white portion of the housing. The brightness of R herein corresponds to the brightness of the red component obtained when a color image of the measurement portion or the white portion captured with a smartphone is decomposed into the three primary colors red®, green (g), and blue (b). In the present example, a blue color former TMB was used. Thus, the exhibited color was quantified based on the amount of decrease in the brightness of the red component. Tables 1 to 3 below show the absorbance×10, the mean value, the standard deviation (SD), and the coefficient of variation (CV). Note that in the following examples, the tables show the values “absorbance×10” instead of the actually measured values of absorbance. This is for the sake of convenience of handling data because the actually measured values were small. However, evaluation in the present invention is not limited to the evaluation based on the values “absorbance×10,” and it is acceptable as long the respective pieces of data can be compared based on the same criterion.

TABLE 1
TMB 0 min

| HRP- | | | | | | |
|---|---|---|---|---|---|---|
| Labeled | | | | | | |
| Antibodies | Absorbance × 10 | | | | | |
| (μg/mL) | First | Second | Third | Ave. | SD | CV(%) |
| 0 | 0.07 | 0.05 | 0.05 | 0.06 | 0.012 | 20 |
| 0.03125 | 0.12 | 0.10 | 0.10 | 0.11 | 0.012 | 11 |
| 0.0625 | 0.13 | 0.13 | 0.10 | 0.12 | 0.017 | 14 |
| 0.125 | 0.16 | 0.12 | 0.10 | 0.13 | 0.031 | 24 |
| 0.25 | 0.14 | 0.03 | 0.06 | 0.09 | 0.042 | 45 |
| 0.5 | 0.11 | 0.02 | 0.03 | 0.05 | 0.049 | 92 |

TABLE 2
TMB 5 min

| HRP- | | | | | | |
|---|---|---|---|---|---|---|
| Labeled | | | | | | |
| Antibodies | Absorbance × 10 | | | | | |
| (μg/mL) | First | Second | Third | Ave. | SD | CV(%) |
| 0 | 0.04 | −0.04 | −0.04 | −0.01 | 0.046 | −346 |
| 0.03125 | 0.90 | 0.51 | 0.77 | 0.73 | 0.199 | 27 |
| 0.0625 | 1.48 | 1.45 | 1.41 | 1.45 | 0.035 | 2 |
| 0.125 | 2.10 | 1.91 | 2.11 | 2.04 | 0.113 | 6 |
| 0.25 | 2.67 | 2.93 | 2.97 | 2.86 | 0.163 | 6 |
| 0.5 | 3.05 | 3.05 | 3.36 | 3.15 | 0.179 | 6 |

A calibration curve, which is obtained by setting the concentration of the HRP-labeled antibodies on the X-axis and setting the absorbance×10 on the Y-axis based on the results in Table 2, is Y=5.722X+0.7778 (coefficient of determination: R²=0.7698).

TABLE 3
TMB 10 min

| HRP- | | | | | | |
|---|---|---|---|---|---|---|
| Labeled | | | | | | |
| Antibodies | Absorbance × 10 | | | | | |
| (μg/mL) | First | Second | Third | Ave. | SD | CV(%) |
| 0 | 0.03 | −0.02 | −0.02 | 0.00 | 0.029 | −866 |
| 0.03125 | 1.25 | 0.75 | 1.14 | 1.05 | 0.263 | 25 |
| 0.0625 | 2.00 | 1.84 | 2.02 | 1.95 | 0.099 | 5 |
| 0.125 | 2.50 | 2.32 | 2.89 | 2.57 | 0.291 | 11 |
| 0.25 | 2.72 | 3.26 | 3.33 | 3.10 | 0.334 | 11 |
| 0.5 | 2.78 | 3.09 | 3.45 | 3.11 | 0.335 | 11 |

A calibration curve, which is obtained by setting the concentration of the HRP-labeled antibodies on the X-axis and setting the absorbance×10 on the Y-axis based on the results in Table 3, is Y=5.1177X+1.1365 (coefficient of determination: R²=0.602).

A signal from the internal standard section 54 in the assay device was visually observed before the addition of the color former and after 10 minutes had elapsed from the addition. Before the addition of the color former, the internal standard section 54 in each of the fluidic channels 1 to 6 exhibited a white color, but after 10 minutes had elapsed from the addition, a stronger blue signal was confirmed as the concentration of the RP-labeled antibodies in the applied liquid was higher among the fluidic channels 1 to 6 (not shown).

### (3) Assay for Known Concentration of Target Substance

Next, a sample containing a preset concentration of allergens as a target substance was applied to the assay device so that the absorbance of each of the detection section and the internal standard section was obtained. As the sample, wheat standard solutions that respectively contain 0 ng/mL, 2.5 ng/mL, 12.5 ng/mL, 25 ng/mL, and 50 ng/mL of wheat were used. The assay device was produced as in (1). Then, in (2), following the suction and cleaning, two droplets of the sample were applied (about 40 μL/droplet). Then, after five minutes had elapsed, five droplets of a cleaning solution were applied (about 40 μL/droplet) to each microfluidic channel to be cleaned. Then, HRP-labeled antibodies, a cleaning solution, and a color former were applied as in (2). In the present experiment, samples each containing the same concentration of allergens were applied to six fluidic channels of a single assay device so that the absorbance of each of the detection sections and the internal standard sections was obtained. Tables 4 to 9 below show the results.

TABLE 4

| | Concen- | | | |
|---|---|---|---|---|
| | tration | Concen- | | Absorbance × |
| | of Internal | tration | Absorbance × | 10 of |
| Fluidic | Standard | of Target | 10 of | Internal |
| Channel | Substance | Substance | Detection | Standard |
| No. | (μg/mL) | (ng/mL) | Section | Section |
| 1 | 0.125 | 0 | 0.02 | 2.33 |
| 2 | | | 0.18 | 2.49 |
| 3 | | | 0.10 | 2.35 |
| 4 | | | 0.08 | 2.45 |
| 5 | | | 0.02 | 2.46 |
| 6 | | | 0.02 | 2.00 |

TABLE 5

| | Concen- | | | |
|---|---|---|---|---|
| | tration | Concen- | | Absorbance × |
| | of Internal | tration | Absorbance × | 10 of |
| Fluidic | Standard | of Target | 10 of | Internal |
| Channel | Substance | Substance | Detection | Standard |
| No. | (μg/mL) | (ng/mL) | Section | Section |
| 1 | 0.125 | 3.125 | 0.30 | 1.97 |
| 2 | | | 0.17 | 2.41 |
| 3 | | | 0.25 | 2.37 |
| 4 | | | 0.23 | 2.64 |
| 5 | | | 0.20 | 2.61 |
| 6 | | | 0.14 | 2.47 |

TABLE 6

| | Concen- | | | |
|---|---|---|---|---|
| | tration | Concen- | | Absorbance × |
| | of Internal | tration | Absorbance × | 10 of |
| Fluidic | Standard | of Target | 10 of | Internal |
| Channel | Substance | Substance | Detection | Standard |
| No. | (μg/mL) | (ng/mL) | Section | Section |
| 1 | 0.125 | 6.25 | 0.15 | 1.75 |
| 2 | | | 0.22 | 1.75 |
| 3 | | | 0.27 | 1.78 |
| 4 | | | 0.30 | 2.16 |
| 5 | | | 0.24 | 2.03 |

TABLE 7

| | Concen- | | | |
|---|---|---|---|---|
| | tration | Concen- | | Absorbance × |
| | of Internal | tration | Absorbance × | 10 of |
| Fluidic | Standard | of Target | 10 of | Internal |
| Channel | Substance | Substance | Detection | Standard |
| No. | (μg/mL) | (ng/mL) | Section | Section |
| 1 | 0.125 | 12.5 | 0.37 | 1.90 |
| 2 | | | 0.41 | 1.89 |
| 3 | | | 0.39 | 2.15 |
| 4 | | | 0.38 | 1.80 |
| 5 | | | 0.33 | 2.10 |
| 6 | | | 0.28 | 1.82 |

TABLE 8

| | Concen- | | | |
|---|---|---|---|---|
| | tration | Concen- | | Absorbance × |
| | of Internal | tration | Absorbance × | 10 of |
| Fluidic | Standard | of Target | 10 of | Internal |
| Channel | Substance | Substance | Detection | Standard |
| No. | (μg/mL) | (ng/mL) | Section | Section |
| 1 | 0.125 | 25 | 0.83 | 2.47 |
| 2 | | | 1.07 | 2.50 |
| 3 | | | 1.07 | 2.56 |
| 4 | | | 0.72 | 2.20 |
| 5 | | | 0.75 | 2.00 |
| 6 | | | 0.67 | 2.04 |

TABLE 9

| | Concen- | | | |
|---|---|---|---|---|
| | tration | Concen- | | Absorbance × |
| | of Internal | tration | Absorbance × | 10 of |
| Fluidic | Standard | of Target | 10 of | Internal |
| Channel | Substance | Substance | Detection | Standard |
| No. | (μg/mL) | (ng/mL) | Section | Section |
| 1 | 0.125 | 50 | 1.26 | 2.14 |
| 2 | | | 1.36 | 2.50 |
| 3 | | | 1.46 | 2.13 |
| 4 | | | 1.28 | 2.28 |
| 5 | | | 1.37 | 1.88 |

As a result of visually observing the signals from the detection sections 14 and the internal standard sections 54, it was found that all of the signals from the internal standard sections had approximately the same degree of blue intensity. Regarding the signals from the detection sections, the higher the concentration of allergens, the stronger the blue signal that was confirmed (not shown).

### (4) Assay of Food Allergens

An assay was performed for the purpose of detecting allergens contained in food. As the samples, the wheat standard solution containing 25 ng/mL wheat used in (3) above, a sample derived from chicken meatballs, and a sample derived from sweet potato cakes were used. The chicken meatballs and the sweet potato cakes were produced by adding wheat powder to raw materials so that the final concentration of wheat proteins became 10 μg/g. Each food was extracted using 20 times the volume of the extract solution, the supernatant was filtered after centrifugation, and the filtrate was diluted with 20 times the volume of the diluent to produce the measurement sample.

An assay was performed as in (3) above except that the foregoing samples were used so that absorbance was obtained. Table 10 below shows the results.

TABLE 10

| | | | | Absor- |
|---|---|---|---|---|
| | | Concentration | Absor- | bance × |
| | | of Internal | bance × | 10 of |
| Fluidic | | Standard | 10 of | Internal |
| Channel | | Substance | Detection | Standard |
| No. | Sample | (μg/mL) | Section | Section |
| 1 | 25 ng/ml of Wheat | 0.25 | 1.05 | 3.20 |
| 2 | 25 ng/ml of Wheat | | 1.04 | 3.65 |
| 3 | Chicken Meatballs | | 1.49 | 3.56 |
| 4 | Chicken Meatballs | | 1.16 | 3.34 |
| 5 | Sweet Potato Cakes | | 1.37 | 2.96 |
| 6 | Sweet Potato Cakes | | 1.37 | 2.51 |

As shown in the results in Table 10, it was possible to perform the measurement of wheat by using the measurement samples extracted from sweet potato cakes and chicken meatballs for the measurement of the actual samples. It was also possible to obtain a constant level of color exhibited in the internal standard section without any inhibition.

### (5) Detection of Defective Assay Device

The layers S2 to S11 as well as the other members to form the assay device were bonded together as shown in FIG. 2 as in (1) except that 20 μL of 20.7 μg/mL anti-allergen antibodies was immobilized as the detection antibodies, and 20 μL of 20 μg/mL anti-mouse IgG antibodies was immobilized as the internal standard substance on the internal standard section. Then, as in (2), blocking and aspiration were performed, and then, the top-side casing layer S1 made of a white ABS resin was bonded to the underlying layers. Cleaning after the bonding, and the application of samples, HRP-labeled antibodies, and a stain were performed through the same procedures as those in (1). As the samples, wheat standard solutions that respectively contain 0 ng/mL, 2.5 ng/mL, 12.5 ng/mL, 25 ng/mL and 50 ng/mL wheat were used, and the concentration of the HRP-labeled antibodies was set to 0.25 μg/mL.

Six assay devices, each including six fluidic channels, were prepared. Wheat standard solutions of 0 ng/mL, 2.5 ng/mL, 12.5 ng/mL, 25 ng/mL, and 50 ng/mL were respectively applied to the fluidic channels 1 to 5 of each assay device so that the absorbance of each of the detection section and the internal standard section was obtained. The absorbance was obtained using a method similar to that in (2) above. From a calibration curve for the internal standard section created in advance, it was found that the minimum detectable value of absorbance obtained when the assay device and the HRP-labeled antibodies are normal is 2.0. Thus, herein, a case in which the absorbance×10 of the internal standard section is 2.0 was set as the threshold. When the detected value was less than or equal to 2.0, it was determined that the detection had failed, and the assay had been abnormal.

### Results of Normal Assay

Table 11 below shows the absorbance, the mean value, the standard deviation (SD), and the coefficient of variation (CV) of the detection section, and the absorbance, the mean value (Ave.), the standard deviation (SD), and the coefficient of variation (CV) of the internal standard section when one of the six assay devices was used.

TABLE 11

| | | | | Absor- |
|---|---|---|---|---|
| | | Concentration | Absor- | bance × |
| | Wheat | of Internal | bance × | 10 of |
| Fluidic | Standard | Standard | 10 of | Internal |
| Channel | Solution | Substance | Detection | Standard |
| No. | (ng/mL) | (μg/mL) | Section | Section |
| 1 | 0 | 0.25 | 0.70 | 2.44 |
| 2 | 2.5 | | 0.96 | 2.24 |
| 3 | 12.5 | | 1.91 | 2.49 |
| 4 | 25 | | 2.52 | 2.20 |
| 5 | 50 | | 3.06 | 2.52 |
| | | | | SD: 0.176 |
| | | | | CV: 7.3% |

When the assay device shown in Table 11 was used, the absorbance×10 of the internal standard section was greater than 2.0 that was the threshold. Accordingly, it was ensured that the absorbance of the detection section shown in Table 11 was a reliable value.

### Results of Abnormal Assay

Table 12 below shows the absorbance, the mean value, the standard deviation (SD), and the coefficient of variation (CV) of the detection section when the two other assay devices were used. Table 13 below shows the absorbance, the mean value (Ave.), the standard deviation (SD), and the coefficient of variation (CV) of the internal standard section when the two other assay devices were used. The concentration of the internal standard substance was set to 0.25 μg/mL.

TABLE 12

| | Wheat | Absorbance × 10 | | | | |
|---|---|---|---|---|---|---|
| | Standard | First | Second | | | |
| Fluidic | Solution | Assay | Assay | | | |
| Channel No. | (ng/mL) | Device | Device | Ave. | SD | CV(%) |
| Fluidic | 0 | 0.60 | 0.73 | 0.67 | 0.092 | 13.8 |
| Channel 1 | | | | | | |
| Fluidic | 2.5 | 0.68 | 0.74 | 0.71 | 0.042 | 6.0 |
| Channel 2 | | | | | | |
| Fluidic | 12.5 | 1.01 | 1.05 | 1.03 | 0.028 | 2.7 |
| Channel 3 | | | | | | |
| Fluidic | 25 | 1.31 | 1.06 | 1.19 | 0.177 | 14.9 |
| Channel 4 | | | | | | |
| Fluidic | 50 | 1.88 | 2.05 | 1.97 | 0.120 | 6.1 |
| Channel 5 | | | | | | |

TABLE 13

| | Absorbance × 10 | | | | |
|---|---|---|---|---|---|
| Internal | First Assay | Second Assay | | | CV |
| Standard Section | Device | Device | Ave. | SD | (%) |
| Fluidic Channel 1 | 0.93 | 1.05 | 0.99 | 0.085 | 8.6 |
| Fluidic Channel 2 | 0.88 | 1.20 | 1.04 | 0.226 | 21.8 |
| Fluidic Channel 3 | 0.95 | 0.86 | 0.91 | 0.064 | 7.0 |
| Fluidic Channel 4 | 0.86 | 0.98 | 0.92 | 0.085 | 9.2 |
| Fluidic Channel 5 | 0.83 | 1.16 | 1.00 | 0.233 | 23.5 |
| Ave. | 0.89 | 1.05 | 0.97 | | |
| SD | 0.049 | 0.137 | | 0.129 | |
| CV(%) | 5.6 | 13.1 | | | 13.3 |

When the two assay devices shown in Tables 12 and 13 were used, the absorbance×10 of the internal standard section in each of the first and second assay devices was less than 2.0 that is the threshold. Accordingly, the absorbance of the detection section shown in Table 12 is not recognized as a reliable value, and it was thus possible to determine that the two assay devices are defective. Examples of the cause of the defect include degradation of the HRP-labeled antibodies and the blocking agent, the immobilized conditions of the detection antibodies and the internal standard substance, and the accuracy of the chip.

(6) Examination of Positional Relationship between Detection Section and Internal Standard Section

### (a) Internal Standard Section on Upstream Side

Three assay devices were produced as in (5), except that in each assay unit, the internal standard section was provided on the upstream side and the detection section was provided on the downstream side, and then, an assay was performed through procedures similar to those in (5). The conditions of the samples and the HRP-labeled antibodies were also set similar to those in (5) so that absorbance was obtained. Table 14 below shows the absorbance, the mean value (Ave.), the standard deviation (SD), and the coefficient of variation (CV) of the detection section. Table 15 below shows the absorbance, the mean value, the standard deviation (SD), and the coefficient of variation (CV) of the internal standard section. An assay device in which the absorbance×10 of the internal standard section was less than or equal to 1.5 was determined to be defective. The numerical value 8.4% below the lower right column of Table 14 represents the mean value of CV.

TABLE 14

| | Wheat | Absorbance × 10 | | | | | |
|---|---|---|---|---|---|---|---|
| Fluidic | Standard | First | Second | Third | | | |
| Channel | Solution | Assay | Assay | Assay | | | |
| No. | (ng/mL) | Device | Device | Device | Ave. | SD | CV(%) |
| Fluidic | 0 | 0.43 | 0.43 | 0.47 | 0.44 | 0.023 | 5.2 |
| Channel 1 | | | | | | | |
| Fluidic | 2.5 | 0.62 | 0.58 | 0.54 | 0.58 | 0.040 | 6.9 |
| Channel 2 | | | | | | | |
| Fluidic | 12.5 | 1.44 | 1.33 | 1.04 | 1.27 | 0.207 | 16.3 |
| Channel 3 | | | | | | | |
| Fluidic | 25 | 1.89 | 2.14 | 1.71 | 1.91 | 0.216 | 11.3 |
| Channel 4 | | | | | | | |
| Fluidic | 50 | 2.65 | 2.64 | 2.75 | 2.68 | 0.061 | 2.3 |
| Channel 5 | | | | | | | |

8.4

TABLE 15

| | Absorbance × 10 | | | | | |
|---|---|---|---|---|---|---|
| Internal | First | Second | Third | | | |
| Standard | Assay | Assay | Assay | | | |
| Section | Device | Device | Device | Ave. | SD | CV(%) |
| Fluidic | 1.78 | 2.02 | 3.05 | 2.28 | 0.675 | 29.5 |
| Channel 1 | | | | | | |
| Fluidic | 1.96 | 2.65 | 2.74 | 2.45 | 0.427 | 17.4 |
| Channel 2 | | | | | | |
| Fluidic | 1.97 | 2.56 | 2.71 | 2.41 | 0.391 | 16.2 |
| Channel 3 | | | | | | |
| Fluidic | 2.02 | 2.47 | 2.75 | 2.41 | 0.368 | 15.3 |
| Channel 4 | | | | | | |
| Fluidic | 2.27 | 2.26 | 2.88 | 2.47 | 0.355 | 14.4 |
| Channel 5 | | | | | | |
| Ave. | 2.00 | 2.39 | 2.83 | 2.41 | | |
| SD | 0.176 | 0.253 | 0.141 | | 0.393 | |
| CV(%) | 8.8 | 10.6 | 5.0 | | | 16.4 |

From the results of Tables 14 and 15, it was confirmed that the quantification of a target substance is possible even when the positional relationship between the detection section and the internal standard section is reversed.

Next, a methylene blue stain solution was flowed through each fluidic channel after completion of measurement so that the absorbance of each of the detection section and the internal standard section was obtained, and the difference in production accuracy between the fluidic channels was confirmed. Table 16 below shows the absorbance, the mean value (Ave.), the standard deviation (SD), and the coefficient of variation (CV) of the detection section. Table 17 below shows the absorbance, the mean value, the standard deviation (SD), and the coefficient of variation (CV) of the internal standard section. Based on the results of the experiments performed in advance, when the absorbance×10 was less than or equal to 1.5, or when CV was greater than or equal to 5.0%, the produced assay device was determined to be defective.

TABLE 16

| | Absorbance × 10 | | | | | |
|---|---|---|---|---|---|---|
| | First | Second | Third | | | |
| Detection | Assay | Assay | Assay | | | |
| Section | Device | Device | Device | Ave. | SD | CV(%) |
| Fluidic | 1.60 | 1.67 | 1.56 | 1.61 | 0.056 | 3.5 |
| Channel 1 | | | | | | |
| Fluidic | 1.62 | 1.66 | 1.56 | 1.61 | 0.050 | 3.1 |
| Channel 2 | | | | | | |
| Fluidic | 1.68 | 1.73 | 1.60 | 1.67 | 0.066 | 3.9 |
| Channel 3 | | | | | | |
| Fluidic | 1.66 | 1.66 | 1.67 | 1.66 | 0.006 | 0.3 |
| Channel 4 | | | | | | |
| Fluidic | 1.62 | 1.67 | 1.72 | 1.67 | 0.050 | 3.0 |
| Channel 5 | | | | | | |
| Ave. | 1.64 | 1.68 | 1.62 | 1.65 | | |
| SD | 0.033 | 0.029 | 0.071 | | 0.051 | |
| CV(%) | 2.0 | 1.8 | 4.4 | | | 3.1 |

TABLE 17

| | Absorbance × 10 | | | | | |
|---|---|---|---|---|---|---|
| Internal | First | Second | Third | | | |
| Standard | Assay | Assay | Assay | | | |
| Section | Device | Device | Device | Ave. | SD | CV(%) |
| Fluidic | 1.72 | 1.67 | 1.64 | 1.68 | 0.040 | 2.4 |
| Channel 1 | | | | | | |
| Fluidic | 1.68 | 1.69 | 1.66 | 1.68 | 0.015 | 0.9 |
| Channel 2 | | | | | | |
| Fluidic | 1.65 | 1.69 | 1.72 | 1.69 | 0.035 | 2.1 |
| Channel 3 | | | | | | |
| Fluidic | 1.76 | 1.68 | 1.71 | 1.72 | 0.040 | 2.4 |
| Channel 4 | | | | | | |
| Fluidic | 1.68 | 1.72 | 1.79 | 1.73 | 0.065 | 3.2 |
| Channel 5 | | | | | | |
| Ave. | 1.70 | 1.69 | 1.70 | 1.70 | | |
| SD | 0.043 | 0.019 | 0.059 | | 0.040 | |
| CV(%) | 2.5 | 1.1 | 3.4 | | | 2.4 |

From the results in Table 16 and Table 17, it was found that CV was less than or equal to 5.0% among the fluidic channels 1 to 5, and also, CV was less than or equal to 5% among the first to third assay devices. This shows that the measurement was performed normally in a state in which the appropriate thickness of each of the microfluidic channels of the assay devices used in Table 16 and Table 17 was maintained. Note that when an assay is performed using a high-concentration sample, if an assay device, which includes the detection section located on the upstream side and the internal standard section located on the downstream side, is used, the color exhibited in the internal standard section may be affected. However, it was confirmed when the internal standard section was disposed on the upstream side, no change was seen in the internal standard section, even if the concentration of wheat in the sample was high (details of the data are not shown).

### (b) Internal Standard Section on Downstream Side

Assay devices were produced as in (a), except that in each assay unit, the internal standard section was provided on the downstream side and the detection section was provided on the upstream side, and then, an assay was performed under conditions and through procedures similar to those in (a). Table 18 below shows the absorbance, the mean value (Ave.), the standard deviation (SD), and the coefficient of variation (CV) of the detection section. Table 19 below shows the absorbance, the mean value (Ave.), the standard deviation (SD), and the coefficient of variation (CV) of the internal standard section. An assay device in which the absorbance×10 of the internal standard section is less than or equal to 1.5 was determined to be defective. The numerical value 6.900 below the lower right column of Table 18 represents the mean value of CV.

TABLE 18

| | Wheat | Absorbance × 10 | | | | | |
|---|---|---|---|---|---|---|---|
| Fluidic | Standard | First | Second | Third | | | |
| Channel | Solution | Assay | Assay | Assay | | | |
| No. | (ng/mL) | Device | Device | Device | Ave. | SD | CV(%) |
| Fluidic | 0 | 0.44 | 0.41 | 0.41 | 0.42 | 0.017 | 4.1 |
| Channel 1 | | | | | | | |
| Fluidic | 2.5 | 0.54 | 0.52 | 0.57 | 0.54 | 0.025 | 4.6 |
| Channel 2 | | | | | | | |
| Fluidic | 12.5 | 1.45 | 1.32 | 1.25 | 1.34 | 0.101 | 7.6 |
| Channel 3 | | | | | | | |
| Fluidic | 25 | 1.88 | 2.02 | 1.45 | 1.78 | 0.297 | 16.7 |
| Channel 4 | | | | | | | |
| Fluidic | 50 | 2.70 | 2.74 | 2.65 | 2.70 | 0.045 | 1.7 |
| Channel 5 | | | | | | | |

6.9

TABLE 19

| | Absorbance × 10 | | | | | |
|---|---|---|---|---|---|---|
| Internal | First | Second | Third | | | |
| Standard | Assay | Assay | Assay | | | |
| Section | Device | Device | Device | Ave. | SD | CV(%) |
| Fluidic | 1.99 | 1.65 | 2.10 | 1.91 | 0.235 | 12.3 |
| Channel 1 | | | | | | |
| Fluidic | 1.91 | 2.38 | 1.95 | 2.08 | 0.261 | 12.5 |
| Channel 2 | | | | | | |
| Fluidic | 2.44 | 2.17 | 1.67 | 2.09 | 0.391 | 18.7 |
| Channel 3 | | | | | | |
| Fluidic | 2.52 | 2.12 | 1.96 | 2.20 | 0.288 | 13.1 |
| Channel 4 | | | | | | |
| Fluidic | 2.30 | 2.51 | 1.64 | 2.15 | 0.454 | 21.1 |
| Channel 5 | | | | | | |
| Ave. | 2.23 | 2.17 | 1.86 | 2.09 | | |
| SD | 0.271 | 0.329 | 0.200 | | 0.301 | |
| CV(%) | 12.1 | 15.2 | 10.7 | | | 14.4 |

Next, each of the fluidic channels after the completion of the measurement (b) was subjected to the analysis of absorbance using a methylene blue stain solution and the evaluation of the difference in production accuracy among the fluidic channels under conditions and through procedures similar to those in (a). Table 20 below shows the absorbance, the mean value (Ave.), the standard deviation (SD), and the coefficient of variation (CV) of the detection section, and Table 21 below shows the absorbance, the mean value (Ave.), the standard deviation (SD), and the coefficient of variation (CV) of the internal standard section. The criterion used to determine if an assay device was defective was similar to that in (a).

TABLE 20

| | Absorbance × 10 | | | | | |
|---|---|---|---|---|---|---|
| | First | Second | Third | | | |
| Detection | Assay | Assay | Assay | | | |
| Section | Device | Device | Device | Ave. | SD | CV(%) |
| Fluidic | 1.18 | 1.76 | 1.70 | 1.76 | 0.055 | 3.1 |
| Channel 1 | | | | | | |
| Fluidic | 1.75 | 1.83 | 1.75 | 1.78 | 0.046 | 2.6 |
| Channel 2 | | | | | | |
| Fluidic | 1.78 | 1.89 | 1.78 | 1.82 | 0.064 | 3.5 |
| Channel 3 | | | | | | |
| Fluidic | 1.81 | 1.87 | 1.77 | 1.82 | 0.050 | 2.8 |
| Channel 4 | | | | | | |
| Fluidic | 1.77 | 1.77 | 1.82 | 1.79 | 0.029 | 1.6 |
| Channel 5 | | | | | | |
| Ave. | 1.78 | 1.82 | 1.76 | 1.79 | | |
| SD | 0.026 | 0.058 | 0.044 | | 0.049 | |
| CV(%) | 1.5 | 3.2 | 2.5 | | | 2.7 |

TABLE 21

| | Absorbance × 10 | | | | | |
|---|---|---|---|---|---|---|
| Internal | First | Second | Third | | | |
| Standard | Assay | Assay | Assay | | | |
| Section | Device | Device | Device | Ave. | SD | CV(%) |
| Fluidic | 1.72 | 1.71 | 1.75 | 1.73 | 0.021 | 1.2 |
| Channel 1 | | | | | | |
| Fluidic | 1.68 | 1.77 | 1.72 | 1.72 | 0.045 | 2.6 |
| Channel 2 | | | | | | |
| Fluidic | 1.77 | 1.86 | 1.71 | 1.78 | 0.075 | 4.2 |
| Channel 3 | | | | | | |
| Fluidic | 1.77 | 1.82 | 1.79 | 1.79 | 0.025 | 1.4 |
| Channel 4 | | | | | | |
| Fluidic | 1.76 | 1.71 | 1.71 | 1.43 | 0.029 | 1.7 |
| Channel 5 | | | | | | |
| Ave. | 1.74 | 1.77 | 1.74 | 1.75 | | |
| SD | 0.039 | 0.067 | 0.034 | | 0.049 | |
| CV(%) | 2.3 | 3.8 | 2.0 | | | 2.8 |

From the results in Tables 20 and 21, it was found that absorbance was greater than or equal 1.5 and CV was less than or equal to 5% in each case. This shows that the measurement was performed normally in a state in which the appropriate thickness of each of the fluidic channels of the assay devices was maintained.

From the results of (a) and (b) above, it was confirmed that according to the assay device of the present invention, normal assays are possible regardless of whether the internal standard section is provided on the upstream side or the downstream side. A conventional device has a problem, in view of general knowledge of immunoassays, in that when the internal standard section is provided on the upstream side, antibodies are first trapped in the internal standard section, and thus, that the concentration of antibodies in the detection section cannot be known, which makes it difficult to control the device. In the device of the present invention, a reaction progresses mainly when a flow of a liquid stops and the liquid thus stays in a microfluidic channel based on the stop-and-flow principle, unlike a reaction that occurs in a mobile phase in immunochromatography. Furthermore, unlike with immunochromatography, since molecular recognition elements, such as antibodies, are two-dimensionally immobilized only on the surface of each microfluidic channel, the frequency of binding of antigens to the antibodies during the liquid flow is low. Therefore, even when the internal standard section is provided on the upstream side, measurement can be performed without the problems of conventional devices. Furthermore, even when compared with a case in which the internal standard section is provided on the downstream side, the accuracy of measurement was found to be the same.

### (7) Examination of Internal Standard Substance

An internal standard substance that does not react with the measurement sample was examined. An assay device to be used was produced as in (1). The assay device used in the present example has the configuration shown in FIG. 1 in which the detection section is located on the upstream side and the internal standard section is located on the downstream side. A test was performed using goat anti-rabbit IgG antibodies as the internal standard substance. 20 μL of 20.7 μg/mL anti-allergen antibodies (antibodies to wheat gliadin) were immobilized as the detection antibodies on the detection section 14 of the assay device, and 20 μL of 20 μg/mL goat anti-rabbit IgG antibodies were immobilized as the internal standard substance on the internal standard section 54. As the sample, a 0 ng/mL or a 2.5 ng/mL wheat standard solution was used, and each sample was applied continuously in two droplets (about 40 μL/droplet). As the labeled antibodies, HRP-labeled anti-allergen antibodies and 0.016 μg/mL RP-labeled rabbit IgG antibodies were applied continuously in two droplets (about 40 μL/droplet). The samples and the labeled antibodies were applied to the assay device as in (2) and (3). As the color former, TMB was applied continuously in three droplets (about 40 μL/droplet). The absorbance of each of the detection section 14 and the internal standard section 54 was analyzed to examine the minimum detectable sensitivity. The 2SD method was used to confirm the detection limit. With the 2SD method, when absorbance+2SD at a given concentration and absorbance −2SD at a concentration higher than that do not overlap, it is possible to determine that there is a significant difference between the two concentrations.

Table 22 below shows, for the sample containing 0 ng/mL wheat, the absorbance, the standard deviation (SD), the coefficient of variation (CV), the mean value (Ave.) −2SD, and Ave. +2SD of the detection section. Table 23 below shows, for the sample containing 2.5 ng/mL wheat, the absorbance, the standard deviation (SD), the coefficient of variation (CV), the mean value (Ave.) −2SD, and Ave. +2SD of the detection section.

TABLE 22

| | Absorbance × 10 | | | | | | | | | |
|---|---|---|---|---|---|---|---|---|---|---|
| Detection | Fluidic | Fluidic | Fluidic | Fluidic | Fluidic | | | CV | Ave. − | Ave. + |
| Section | Channel 1 | Channel 2 | Channel 3 | Channel 4 | Channel 5 | Ave. | SD | (%) | 25 D | 25 D |
| First Assay | 0.63 | 0.61 | 0.62 | 0.62 | 0.62 | 0.62 | 0.005 | 0.8 | 0.61 | 0.63 |
| Device | | | | | | | | | | |
| Second Assay | 0.64 | 0.66 | 0.64 | 0.66 | 0.67 | 0.65 | 0.014 | 2.2 | 0.63 | 0.68 |
| Device | | | | | | | | | | |
| Ave. | 0.63 | 0.64 | 0.63 | 0.64 | 0.65 | 0.64 | | | 0.60 | 0.68 |
| SD | 0.008 | 0.036 | 0.013 | 0.028 | 0.032 | | 0.020 | | | |
| CV(%) | 1.3 | 5.7 | 2.1 | 4.4 | 4.9 | | | 3.2 | | |

TABLE 23

| | Absorbance × 10 | | | | | | | | | |
|---|---|---|---|---|---|---|---|---|---|---|
| Detection | Fluidic | Fluidic | Fluidic | Fluidic | Fluidic | | | CV | Ave. − | Ave. + |
| Section | Channel 1 | Channel 2 | Channel 3 | Channel 4 | Channel 5 | Ave. | SD | (%) | 25 D | 25 D |
| First Assay | 0.94 | 0.98 | 0.95 | 1.06 | 1.05 | 1.00 | 0.056 | 5.6 | 0.88 | 1.11 |
| Device | | | | | | | | | | |
| Second Assay | 0.99 | 1.01 | 0.99 | 1.01 | 0.94 | 0.99 | 0.030 | 3.1 | 0.93 | 1.05 |
| Device | | | | | | | | | | |
| Ave. | 0.96 | 1.00 | 0.97 | 1.03 | 0.99 | 0.99 | | | 0.91 | 1.08 |
| SD | 0.034 | 0.019 | 0.031 | 0.033 | 0.082 | | 0.043 | | | |
| CV(%) | 3.5 | 1.9 | 3.2 | 3.1 | 8.2 | | | 4.3 | | |

Table 24 below shows, for the sample containing 0 ng/mL wheat, the absorbance, the standard deviation (SD), and the coefficient of variation (CV) of the internal standard section, and Table 25 below shows, for the sample containing 2.5 ng/mL wheat, the absorbance, the standard deviation (SD), and the coefficient of variation (CV) of the internal standard section.

TABLE 24

| Internal | Absorbance × 10 | | | | | | | |
|---|---|---|---|---|---|---|---|---|
| Standard | Fluidic | Fluidic | Fluidic | Fluidic | Fluidic | | | |
| Section | Channel 1 | Channel 2 | Channel 3 | Channel 4 | Channel 5 | Ave. | SD | CV(%) |
| First Assay | 3.11 | 3.12 | 2.84 | 3.16 | 3.44 | 3.13 | 0.212 | 6.8 |
| Device | | | | | | | | |
| Second Assay | 2.73 | 2.79 | 2.82 | 2.66 | 2.56 | 2.71 | 0.103 | 3.8 |
| Device | | | | | | | | |
| Ave. | 2.92 | 2.96 | 2.83 | 2.91 | 3.00 | 2.92 | | |
| SD | 0.271 | 0.236 | 0.018 | 0.350 | 0.621 | | 0.273 | |
| CV(%) | 9.3 | 8.0 | 0.6 | 12.0 | 20.7 | | | 9.3 |

TABLE 25

| Internal | Absorbance × 10 | | | | | | | |
|---|---|---|---|---|---|---|---|---|
| Standard | Fluidic | Fluidic | Fluidic | Fluidic | Fluidic | | | |
| Section | Channel 1 | Channel 2 | Channel 3 | Channel 4 | Channel 5 | Ave. | SD | CV(%) |
| First Assay | 2.94 | 2.96 | 3.22 | 3.03 | 2.92 | 3.01 | 0.123 | 4.1 |
| Device | | | | | | | | |
| Second Assay | 2.95 | 3.19 | 3.40 | 3.37 | 3.24 | 3.23 | 0.180 | 5.6 |
| Device | | | | | | | | |
| Ave. | 2.95 | 3.07 | 3.31 | 3.20 | 3.08 | 3.12 | | |
| SD | 0.007 | 0.162 | 0.128 | 0.239 | 0.225 | | 0.185 | |
| CV(%) | 0.2 | 5.3 | 3.9 | 7.5 | 7.3 | | | 5.9 |

For the purpose of comparison with the goat anti-rabbit IgG antibodies, 20 μL of 10 μg/mL anti-mouse IgG antibodies was immobilized as the internal standard substance on the internal standard section 54. As the labeled antibodies, 0.125 μg/mL HRP-labeled anti-allergen antibodies (antibodies to wheat gliadin) were used. The samples and the labeled antibodies were applied to the assay device as in (3), and the absorbance of each of the detection section 14 and the internal standard section 54 was analyzed to examine the minimum detectable sensitivity.

Table 26 below shows, for the sample containing 0 ng/mL wheat, the absorbance, the standard deviation (SD), the coefficient of variation (CV), the mean value (Ave.) −2SD, and Ave. +2SD of the detection section, and Table 27 below shows, for the sample containing 2.5 ng/mL wheat, the absorbance, the standard deviation (SD), the coefficient of variation (CV), the mean value (Ave.) −2SD, and Ave. +2SD of the detection section.

TABLE 26

| | Absorbance x 10 | | | | | | | | | |
|---|---|---|---|---|---|---|---|---|---|---|
| | Fluidic | Fluidic | Fluidic | Fluidic | Fluidic | | | | | |
| Detection | Channel | Channel | Channel | Channel | Channel | | | CV | Ave. | Ave. |
| Section | 1 | 2 | 3 | 4 | 5 | Ave. | SD | (%) | -2SD | +2SD |
| First Assay | 0.66 | 0.67 | 0.71 | 0.64 | 0.68 | 0.67 | 0.024 | 3.6 | 0.62 | 0.72 |
| Device | | | | | | | | | | |
| Second Assay | 0.62 | 0.60 | 0.61 | 0.61 | 0.62 | 0.61 | 0.011 | 1.7 | 0.59 | 0.63 |
| Device | | | | | | | | | | |
| Ave. | 0.64 | 0.63 | 0.66 | 0.62 | 0.65 | 0.64 | | | 0.57 | 0.71 |
| SD | 0.032 | 0.053 | 0.065 | 0.024 | 0.037 | | 0.036 | | | |
| CV(%) | 4.9 | 8.3 | 9.9 | 3.8 | 5.7 | | | 5.6 | | |

TABLE 27

| | Absorbance x 10 | | | | | | | | | |
|---|---|---|---|---|---|---|---|---|---|---|
| Detection | Fluidic | Fluidic | Fluidic | Fluidic | Fluidic | | | CV | Ave. − | Ave. + |
| Section | Channel 1 | Channel 2 | Channel 3 | Channel 4 | Channel 5 | Ave. | SD | (%) | 25 D | 25 D |
| First Assay | 0.88 | 0.78 | 0.85 | 0.86 | 0.86 | 0.85 | 0.039 | 4.6 | 0.77 | 0.92 |
| Device | | | | | | | | | | |
| Second Assay | 0.79 | 0.83 | 0.88 | 0.85 | 0.92 | 0.85 | 0.050 | 5.9 | 0.75 | 0.95 |
| Device | | | | | | | | | | |
| Ave. | 0.83 | 0.80 | 0.87 | 0.85 | 0.89 | 0.85 | | | 0.76 | 0.93 |
| SD | 0.065 | 0.034 | 0.023 | 0.010 | 0.045 | | 0.043 | | | |
| CV(%) | 7.7 | 4.2 | 2.7 | 1.2 | 5.0 | | | 5.0 | | |

Table 28 below shows, for the sample containing 0 ng/mL wheat, the absorbance, the standard deviation (SD), and the coefficient of variation (CV) of the internal standard section, and Table 29 below shows, for the sample containing 2.5 ng/mL wheat, the absorbance, the standard deviation (SD), and the coefficient of variation (CV) of the internal standard section.

TABLE 28

| Internal | Absorbance x 10 | | | | | | | |
|---|---|---|---|---|---|---|---|---|
| Standard | Fluidic | Fluidic | Fluidic | Fluidic | Fluidic | | | |
| Section | Channel 1 | Channel 2 | Channel 3 | Channel 4 | Channel 5 | Ave. | SD | CV(%) |
| First Assay | 4.03 | 3.55 | 3.13 | 4.53 | 5.38 | 4.12 | 0.876 | 21.3 |
| Device | | | | | | | | |
| Second Assay | 4.38 | 4.06 | 4.61 | 4.27 | 3.73 | 4.21 | 0.332 | 7.9 |
| Device | | | | | | | | |
| Ave. | 4.21 | 3.81 | 3.87 | 4.40 | 4.56 | 4.17 | | |
| SD | 0.250 | 0.362 | 1.045 | 0.179 | 1.166 | | 0.626 | |
| CV(%) | 5.9 | 9.5 | 27.0 | 4.1 | 25.6 | | | 15.0 |

TABLE 29

| Internal | Absorbance × 10 | | | | | | | |
|---|---|---|---|---|---|---|---|---|
| Standard | Fluidic | Fluidic | Fluidic | Fluidic | Fluidic | | | |
| Section | Channel 1 | Channel 2 | Channel 3 | Channel 4 | Channel 5 | Ave. | SD | CV(%) |
| First Assay | 3.86 | 3.37 | 4.62 | 4.55 | 4.63 | 4.20 | 0.568 | 13.5 |
| Device | | | | | | | | |
| Second Assay | 4.29 | 3.48 | 3.20 | 3.45 | 3.28 | 3.54 | 0.436 | 12.3 |
| Device | | | | | | | | |
| Ave. | 4.07 | 3.42 | 3.91 | 4.00 | 3.95 | 3.87 | | |
| SD | 0.307 | 0.077 | 1.006 | 0.778 | 0.954 | | 0.593 | |
| CV(%) | 7.5 | 2.2 | 25.8 | 19.4 | 24.1 | | | 15.3 |

From the foregoing results, it was confirmed that the values of CV in an assay performed with goat anti-rabbit IgG antibodies are less than those in an assay performed with anti-mouse IgG antibodies. Consequently, it was found that goat anti-rabbit IgG antibodies that do not react with the target substance to be detected are more suitable as the internal standard substance than anti-mouse IgG antibodies that react with the target substance to be detected.

(8) Creation of Standard Curve Using HRP-labeled Rabbit IgG Antibodies (0.016 μg/mL)

An assay device including a detection section located on the upstream side and an internal standard section located on the downstream side, was produced as in (7). 10 μL of 20.7 μg/mL anti-allergen antibodies (antibodies to wheat gliadin) were immobilized as the detection antibodies on the detection section 14, and 10 μL of 20 μg/mL goat anti-rabbit IgG antibodies were immobilized as the internal standard substance on the internal standard section 54. As the samples, 0 ng/mL, 2.5 ng/mL, 12.5 ng/mL, 25 ng/mL, and 50 ng/mL wheat standard solutions were used, and each sample was applied continuously in two droplets (about 40 μL/droplet). As the labeled antibodies, 0.016 μg/mL RP-labeled rabbit IgG antibodies were used, and were applied continuously in two droplets (about 40 μL/droplet). The samples and the labeled antibodies were applied to the assay device as in (2) and (3). As the color former, TMB was applied continuously in three droplets (about 40 μL/droplet). The present experiment was repeated twice.

Table 30 below shows the brightness of R, the standard deviation (SD), the coefficient of variation (CV), the mean value (Ave.) −2SD, and Ave. +2SD of the detection section in the first experiment, and Table 31 below shows the brightness of R, the standard deviation (SD), and the coefficient of variation (CV) of the internal standard section in the first experiment.

TABLE 30

| Wheat | Brightness of R | | | | | | | | | |
|---|---|---|---|---|---|---|---|---|---|---|
| Standard | First | Second | Third | Fourth | Fifth | | | | | |
| Solution | Assay | Assay | Assay | Assay | Assay | | | CV | Ave. − | Ave. + |
| (ng/mL) | Device | Device | Device | Device | Device | Ave. | SD | (%) | 25 D | 25 D |
| 0 | 210.46 | 204.14 | 211.75 | 205.16 | 198.67 | 206.04 | 5.264 | 2.6 | 195.51 | 216.56 |
| 2.5 | 193.83 | 189.57 | 200.93 | 200.21 | 201.67 | 197.24 | 5.304 | 2.7 | 186.63 | 207.85 |
| 12.5 | 172.46 | 155.03 | 157.80 | 170.90 | 167.58 | 164.75 | 7.875 | 4.8 | 149.00 | 180.50 |
| 25 | 120.53 | 112.31 | 131.77 | 111.97 | 137.69 | 122.85 | 11.561 | 9.4 | 99.73 | 145.98 |
| 50 | 96.33 | 91.55 | 105.86 | 106.60 | 114.04 | 102.88 | 8.924 | 8.7 | 85.03 | 120.72 |

TABLE 31

| | Brightness of R | | | | | | | |
|---|---|---|---|---|---|---|---|---|
| Internal | First | Second | Third | Fourth | Fifth | | | |
| Standard | Assay | Assay | Assay | Assay | Assay | | | |
| Section | Device | Device | Device | Device | Device | Ave. | SD | CV(%) |
| Fluidic | 117.27 | 105.77 | 131.83 | 120.86 | 103.46 | 115.84 | 11.593 | 10.0 |
| Channel 1 | | | | | | | | |
| Fluidic | 111.19 | 114.09 | 106.32 | 103.98 | 99.23 | 106.96 | 5.869 | 5.5 |
| Channel 2 | | | | | | | | |
| Fluidic | 107.04 | 109.45 | 108.30 | 106.18 | 101.89 | 106.57 | 2.897 | 2.7 |
| Channel 3 | | | | | | | | |
| Fluidic | 115.63 | 107.47 | 112.60 | 112.71 | 103.92 | 110.47 | 4.693 | 4.2 |
| Channel 4 | | | | | | | | |
| Fluidic | 106.99 | 107.87 | 109.83 | 102.19 | 107.98 | 106.97 | 2.866 | 2.7 |
| Channel 5 | | | | | | | | |
| Ave. | 111.62 | 108.93 | 113.78 | 109.18 | 103.30 | 109.36 | | |
| SD | 4.759 | 3.168 | 10.350 | 7.645 | 3.196 | | 6.898 | |
| CV(%) | 4.3 | 2.9 | 9.1 | 7.0 | 3.1 | | | 6.3 |

From Table 31, a standard curve represented by the following expression was obtained by setting the concentration of the wheat standard solution on the X-axis and setting the brightness of R on the Y-axis.

Y=d+(a−d)/(1+(X/c)\hat{ }b)
a=202.60686, b=2.27938, c=16.09755, d=94.87495, R²=0.9965

In the second experiment, the brightness of R, the standard deviation (SD), the coefficient of variation (CV), the mean value (Ave.) −2SD, and Ave. +2SD of each of the detection section and the internal standard section to which a wheat standard solution was applied were similarly determined (data not shown). Consequently, a standard curve was obtained with accuracy similar to that in the first experiment.

### (9) Correction of Master Calibration Curve Using Positive Control

An assay for correcting the master calibration curve was performed by applying a positive control to two of the fluidic channels of the assay device. The assay device used in the present example has a configuration in which the internal standard section is located on the upstream side and the detection section is located on the downstream side, and was produced as in (1). 20 μL of 20.7 μg/mL anti-allergen antibodies (antibodies to wheat gliadin) were immobilized as the detection antibodies on the detection section, and 20 μL of 20 μg/mL goat anti-rabbit IgG antibodies were immobilized as the internal standard substance on the internal standard section. As the sample, a 0, 2.5, 12.5, 25, or 50 ng/mL wheat standard solution was used, and each sample was applied continuously in two droplets (about 40 μL/droplet). As the labeled antibodies, 0.125 μg/mL HRP-labeled anti-allergen antibodies and 0.016 μg/mL RP-labeled rabbit IgG antibodies were used, and such antibodies were each applied continuously in two droplets (about 40 μL/droplet). The samples and the labeled antibodies were applied to the assay device as in (2) and (3). As the color former, TMB was applied continuously in three droplets (about 40 μL/droplet). The assay device for creating the calibration curve and the assay device for measuring the actual sample have the same configuration, and were produced on the same date. In addition, the wheat standard solutions, the RP-labeled anti-allergen antibodies, and the RP-labeled rabbit IgG antibodies were all prepared on the same date, and were used for experiments on the same date.

First, a 0, 2.5, 12.5, 25, or 50 ng/mL wheat standard solution was applied to the assay device so that absorbance was measured and a calibration curve was created. The results of absorbance measurements are omitted, but the detection limit of the detection section was confirmed to have a significant difference by the 2SD method. From the results of the measurement of the absorbance, a calibration curve represented by the following expression was obtained by setting the concentration of wheat on the X-axis and setting the absorbance×10 on the Y-axis.

Y=d+(a−d)/(1+(X/c)\hat{ }b)
a=0.69037, b=1.68536, c=24.78798, d=4.74334, R²=0.99981

Next, an assay of the concentration of wheat in a food extract was performed using assay devices. Among five fluidic channels of each assay device, a first positive control (a 2.5 ng/mL wheat standard solution) was flowed through a fluidic channel 1, and a second positive control (a 25 ng/mL wheat standard solution) was flowed through a fluidic channel 2. A food extract containing a known concentration of wheat was flowed as the actual sample through fluidic channels 3 to 5. The actual sample with the same concentration was flowed through the fluidic channels 3 to 5 of a single assay device. Three types of actual samples with wheat concentrations of 0 ng/mL, 2.5 ng/mL, and 25 ng/mL, respectively, were applied to the different assay devices to perform tests.

The absorbance of each of the detection sections in the fluidic channels 1 to 5 was measured, and a deviation rate was calculated based on a master calibration curve obtained in advance. In addition, a deviation constant was calculated from the deviation rate, and corrected absorbance was calculated based on the deviation constant. In the present example, the deviation rate and the corrected absorbance are defined as follows. The deviation constant herein is the mean value of the deviation rates obtained from the results of the measurement of the first and second positive controls.

Deviation rate=absorbance (positive control)/absorbance (calibration curve)

Corrected absorbance=the absorbance of the actual sample/the deviation constant

In the present experiment, the absorbance of the actual sample is the mean value of the absorbances of the fluidic channels 3 to 5. The corrected concentration of wheat was calculated based on the corrected absorbance and the master calibration curve.

Table 32 below shows the results of the measurement and calculation for the actual sample with a wheat concentration of 2.5 ng/mL, and Table 33 below shows the results of the measurement and calculation for the actual sample with a wheat concentration of 25 ng/mL. In the present experiment, absorbance is also indicated as the value of “Absorbance×10.” The same experiment was repeated twice, and the food extracts used are indicated as “Food Extract 1” and “Food Extract 2.” In the tables, the recovery rate is defined as follows.

Recovery rate=the corrected concentration of wheat/the concentration of wheat in the actual sample

TABLE 32

| | | | | | | Actual Sample | | | | | |
|---|---|---|---|---|---|---|---|---|---|---|---|
| | Positive Control | | | | | Uncorrected | | | Corrected | | |
| | Absorbance × 10 | | Deviation Rate | | | | Concentration | | | Concentration | |
| | 2.5 | 2.5 | 2.5 | 2.5 | Deviation | Absorbance × | of Wheat | Recovery | Absorbance × | of Wheat | Recovery |
| | ng/ml | ng/mL | ng/ml | ng/ml | Constant | 10 (Ave.) | (ng/ml) | Rate | 10 (Ave.) | (ng/mL) | Rate |
| Food | 0.84 | 2.67 | 1.09 | 0.98 | 1.03 | 0.79 | 2.72 | 108.9% | 0.76 | 2.28 | 91.2% |
| Extract 1 | | | | | | | | | | | |
| Food | 0.77 | 2.6 | 1.00 | 0.95 | 0.98 | 0.77 | 2.51 | 100.5% | 0.79 | 2.84 | 113.4% |
| Extract 2 | | | | | | | | | | | |

TABLE 33

| | | | | | | Actual Sample | | | | | |
|---|---|---|---|---|---|---|---|---|---|---|---|
| | Positive Control | | | | | Uncorrected | | | Corrected | | |
| | Absorbance × 10 | | Deviation Rate | | | | Concentration | | | Concentration | |
| | 2.5 | 2.5 | 2.5 | 2.5 | Deviation | Absorbance × | of Wheat | Recovery | Absorbance × | of Wheat | Recovery |
| | ng/mL | ng/ml | ng/mL | ng/ml | Constant | 10 (Ave.) | (ng/ml) | Rate | 10 (Ave.) | (ng/ml) | Rate |
| Food | 0.79 | 2.26 | 1.03 | 0.83 | 0.93 | 2.12 | 17.30 | 69.2% | 2.29 | 19.20 | 76.8% |
| Extract 1 | | | | | | | | | | | |
| Food | 0.86 | 2.65 | 1.11 | 0.97 | 1.04 | 2.49 | 21.70 | 86.8% | 2.39 | 20.49 | 82.0% |
| Extract 2 | | | | | | | | | | | |

### (10) Detection of Saturation

It was confirmed that saturation of the target substance can be detected by measuring changes in absorbance over time after the color former TMB is added. An assay device, which includes a detection section located on the upstream side and an internal standard section located on the downstream side, was produced as in (1). 10 μL of 20.7 μg/mL anti-allergen antibodies (antibodies to wheat gliadin) were immobilized as the detection antibodies on the detection section 14, and 10 μL of 20 μg/mL goat anti-rabbit IgG antibodies were immobilized as the internal standard substance on the internal standard section 54. For the sample, a wheat sample with a concentration of 1/100,000 times to 1/10 times was used, and as the labeled antibodies, 0.016 μg/mL HRP-labeled rabbit IgG antibodies were used. The magnification herein refers to the dilution factor for an undiluted wheat extract. The samples and the labeled antibodies were applied to the assay device as in (2) and (3). As the color former, TMB was applied continuously in three droplets (about 40 μL/droplet). The brightness of R was measured once every minute starting from the time point of completion of dropping of the color former. Table 34 below shows changes in the brightness over time when the concentration of wheat is 1/10,000 times. Table 35 below shows changes in the brightness over time when the concentration of wheat is 1/1,000 times. Table 36 below shows changes in the brightness over time when the concentration of wheat is 1/100 times.

TABLE 34

| | | Time (min) | | | | | | | | | |
|---|---|---|---|---|---|---|---|---|---|---|---|
| Absorbance | | 1 | 2 | 3 | 4 | 5 | 6 | 7 | 8 | 9 | 10 |
| Fluidic | Absorbance × 10 | 0.91 | 1.17 | 1.44 | 1.67 | 1.92 | 2.19 | 2.44 | 2.69 | 2.96 | 3.12 |
| Channel 1 | Difference | | 0.26 | 0.27 | 0.23 | 0.25 | 0.27 | 0.25 | 0.24 | 0.28 | 0.15 |
| Fluidic | Absorbance × 10 | 0.92 | 1.15 | 1.38 | 1.63 | 1.83 | 2.05 | 2.26 | 2.48 | 2.65 | 2.83 |
| Channel 2 | Difference | | 0.23 | 0.24 | 0.24 | 0.21 | 0.21 | 0.22 | 0.22 | 0.17 | 0.17 |
| Fluidic | Absorbance × 10 | 0.88 | 1.10 | 1.33 | 1.55 | 1.79 | 2.02 | 2.26 | 2.46 | 2.70 | 2.85 |
| Channel 3 | Difference | | 0.22 | 0.23 | 0.22 | 0.24 | 0.23 | 0.23 | 0.20 | 0.24 | 0.15 |
| Fluidic | Absorbance × 10 | 1.21 | 1.62 | 1.99 | 2.35 | 2.68 | 3.01 | 3.35 | 3.65 | 3.91 | 4.12 |
| Channel 4 | Difference | | 0.41 | 0.37 | 0.36 | 0.33 | 0.33 | 0.34 | 0.30 | 0.26 | 0.21 |
| Fluidic | Absorbance × 10 | 1.01 | 1.29 | 1.54 | 1.82 | 2.06 | 2.33 | 2.62 | 2.82 | 3.05 | 3.30 |
| Channel 5 | Difference | | 0.28 | 0.25 | 0.27 | 0.24 | 0.27 | 0.28 | 0.21 | 0.22 | 0.25 |

TABLE 35

| | | Time (min) | | | | | | | | | |
|---|---|---|---|---|---|---|---|---|---|---|---|
| Absorbance | | 1 | 2 | 3 | 4 | 5 | 6 | 7 | 8 | 9 | 10 |
| Fluidic | Absorbance × 10 | 3.69 | 4.85 | 5.40 | 5.51 | 5.35 | 5.06 | 4.76 | 4.50 | 4.26 | 4.05 |
| Channel 1 | Difference | | 1.16 | 0.55 | 0.11 | −0.16 | −0.29 | −0.30 | −0.26 | −0.24 | −0.21 |
| Fluidic | Absorbance × 10 | 4.30 | 5.83 | 6.52 | 6.54 | 6.22 | 5.73 | 5.23 | 4.92 | 4.63 | 4.43 |
| Channel 2 | Difference | | 1.53 | 0.69 | 0.01 | −0.31 | −0.49 | −0.50 | −0.31 | −0.28 | −0.20 |
| Fluidic | Absorbance × 10 | 3.64 | 5.22 | 5.86 | 6.44 | 6.52 | 6.39 | 6.05 | 5.72 | 5.39 | 5.05 |
| Channel 3 | Difference | | 1.58 | 0.64 | 0.57 | 0.09 | −0.13 | −0.34 | −0.33 | −0.33 | −0.34 |
| Fluidic | Absorbance × 10 | 4.39 | 5.86 | 6.58 | 6.53 | 6.09 | 5.60 | 5.16 | 4.76 | 4.49 | 4.32 |
| Channel 4 | Difference | | 1.47 | 0.73 | −0.05 | −0.44 | −0.49 | −0.44 | −0.39 | −0.27 | −0.17 |
| Fluidic | Absorbance × 10 | 3.26 | 4.40 | 5.23 | 5.75 | 6.01 | 6.00 | 5.83 | 5.59 | 5.30 | 5.09 |
| Channel 5 | Difference | | 1.14 | 0.83 | 0.52 | 0.26 | −0.01 | −0.18 | −0.23 | −0.29 | −0.20 |

TABLE 36

| | | Time (min) | | | | | | | | | |
|---|---|---|---|---|---|---|---|---|---|---|---|
| Absorbance | | 1 | 2 | 3 | 4 | 5 | 6 | 7 | 8 | 9 | 10 |
| Fluidic | Absorbance × 10 | 4.75 | 5.28 | 4.75 | 4.11 | 3.66 | 3.38 | 3.14 | 2.90 | 2.78 | 2.65 |
| Channel 1 | Difference | | 0.54 | −0.53 | −0.64 | −0.44 | −0.28 | −0.25 | −0.23 | −0.12 | −0.13 |
| Fluidic | Absorbance × 10 | 3.44 | 4.67 | 5.43 | 5.74 | 5.76 | 5.46 | 4.99 | 4.61 | 4.23 | 3.96 |
| Channel 2 | Difference | | 1.23 | 0.76 | 0.31 | 0.02 | −0.29 | −0.47 | −0.38 | −0.38 | −0.28 |
| Fluidic | Absorbance × 10 | 5.17 | 6.42 | 6.19 | 5.49 | 4.79 | 4.31 | 3.98 | 3.75 | 3.54 | 3.37 |
| Channel 3 | Difference | | 1.25 | −0.23 | −0.70 | −0.69 | −0.48 | −0.33 | −0.22 | −0.21 | −0.17 |
| Fluidic | Absorbance × 10 | 3.25 | 4.36 | 5.17 | 5.74 | 5.95 | 5.89 | 5.53 | 5.19 | 4.79 | 4.43 |
| Channel 4 | Difference | | 1.11 | 0.81 | 0.57 | 0.21 | −0.05 | −0.36 | −0.34 | −0.40 | −0.36 |
| Fluidic | Absorbance × 10 | 4.38 | 5.18 | 5.02 | 4.57 | 4.09 | 3.71 | 3.47 | 3.29 | 3.14 | 3.01 |
| Channel 5 | Difference | | 0.79 | −0.16 | −0.45 | −0.47 | −0.38 | −0.24 | −0.18 | −0.15 | −0.13 |

From the results of the measurement of the sample with a wheat concentration of 1/10,000 times, it was found that the absorbance of each of the fluidic channels 1 to 5 monotonically increased until 10 minutes had elapsed from the start of a reaction, and it did not decrease. From the results of the measurement of the sample with a wheat concentration of 1/1,000 times, it was confirmed that the absorbance of each of the fluidic channels 1 to 5 increased after the start of a reaction, and then, the absorbance of each of the fluidic channels 1 and 2 decreased after five minutes had elapsed, the absorbance of each of the fluidic channels 3 and 5 decreased after six minutes had elapsed, and the absorbance of the fluidic channel 4 decreased after four minutes had elapsed. From the results of the measurement of the sample with a wheat concentration of 1/100 times, it was confirmed that the absorbance of each of the fluidic channels 1 to 5 increased after the start of a reaction, and then, the absorbance of each of the fluidic channels 1, 3, and 5 decreased after three minutes had elapsed, and the absorbance of each of the fluidic channels 2 and 4 decreased after six minutes had elapsed. From such results, it was confirmed that supersaturation did not occur in the sample with a wheat concentration of 1/10,000 times, whereas supersaturation occurred with the sample with a wheat concentration of 1/1,000 times and the sample with a wheat concentration of 1/100 times. Although specific data is not shown, it was confirmed that supersaturation did not occur in the sample with a wheat concentration of 1/100,000 times, whereas supersaturation occurred in the sample with a wheat concentration of 1/10 times. From such results, it was confirmed that supersaturation did not occur with the sample with a wheat concentration of 1/10,000 times, whereas supersaturation occurred with the sample with a wheat concentration of 1/1,000 times and the sample with a wheat concentration of 1/100 times by determining that the measurement was performed normally when absorbance monotonically increased until 1 to 10 minutes had elapsed from the start of a reaction, and determining that supersaturation occurred when absorbance decreased two times in succession during the measurement.

### REFERENCE SIGNS LIST

1 Microfluidic channel
1a One end
1b Other end
2 First porous absorbing medium
3 Separation space
4 Housing space
5 Inlet
6 Parallel ventilation passage
7 Connecting ventilation passage
8 Microfluidic channel wall
8a Top portion
8b Bottom portion
9 Separation space wall
9a Top portion
9b Bottom portion
10 Guide wall
14 Detection section
54 Internal standard section
100 Assay unit

1. An assay device comprising a plurality of assay units, each assay unit comprising:
a microfluidic channel configured to allow a liquid to flow;
a porous absorbing medium disposed at a distance from one end of the microfluidic channel, the one end being located on one side in a flow direction of the liquid; and
a separation space disposed between the one end of the microfluidic channel and the porous absorbing medium,
wherein:
the microfluidic channel comprises, in the microfluidic channel, a detection section having immobilized thereon a substance capable of specifically reacting with a target substance, and an internal standard section having immobilized thereon an internal standard substance, the internal standard section is provided at a distance from the detection section on an upstream or downstream side in the flow direction with respect to the detection section, and the distance between the internal standard section and the detection section is equal to or greater than the width of the microfluidic channel, and
each assay unit comprises two parallel ventilation passages that are respectively adjacent to both sides of the microfluidic channel in a width direction orthogonal to the flow direction, the two parallel ventilation passages communicating with the microfluidic channel to allow for air circulation.

2. (canceled)

3. The assay device according to claim 1, wherein each assay unit further comprises:
an inlet disposed at another end of the microfluidic channel located on another side in the flow direction, the inlet allowing the liquid to be supplied to the microfluidic channel; and
a connecting ventilation passage connecting the two parallel ventilation passages and extending around the inlet to allow for air circulation.

4. The assay device according to claim 1, wherein each assay unit comprises:
a housing space housing the porous absorbing medium;
a separation space wall defining the separation space in cooperation with the porous absorbing medium, the separation space wall comprising a top portion and a bottom portion defining the separation space on both sides in a height direction orthogonal to the flow direction and the width direction; and
a guide wall protruding to the one side in the flow direction from the top portion or the bottom portion of the separation space wall in the housing space,
wherein:
the guide wall abuts the porous absorbing medium in the height direction, and
the top portion or the bottom portion of the separation space wall, and the guide wall are formed to be away from the microfluidic channel in the height direction toward the one side from the other side in the flow direction.

5. An assay method using the assay device according to claim 1, comprising the following steps that are sequentially performed:
(a) applying a sample to the microfluidic channel;
(b) applying a cleaning solution to the microfluidic channel; and
(c) applying a liquid to the microfluidic channel, the liquid comprising a first label capable of specifically binding to a target substance, and a second label capable of specifically binding to the internal standard substance.

6. The assay method according to claim 5, further comprising a step (d) of measuring a signal of the first label in the detection section and a signal of the second label in the internal standard section.

7. The method according to claim 6, wherein the first label and the second label are identical.

8. The assay method according to claim 6, wherein
the first label and the second label are different, and
the method further comprises steps of:
(e) determining, based on a calibration curve of the internal standard substance obtained in advance, and the signal of the second label obtained in the step (d), a deviation rate with respect to the calibration curve of the internal standard substance; and
(f) obtaining, based on the deviation rate, the signal of the first label obtained in the step (d), and a calibration curve of the target substance obtained in advance, a corrected concentration of the target substance.

9. The assay method according to claim 6, further comprising a step (g) of determining if each assay unit is defective based on the signal of the second label obtained in the step (d).

10. The assay method according to claim 6, wherein the step (d) further comprises steps of:
measuring the signal of the first label in the detection section over time, and
determining if detection has failed based on changes in the signal over time.
